(19) United States
(12) Patent Application Publication
(10) Pub. No.: US 2015/0283147 A1
(43) Pub. Date: Oct. 8, 2015

(54) TREATING POLYCYSTIC KIDNEY DISEASE WITH HSP90 INHIBITORY COMPOUNDS

(71) Applicants: SYNTA PHARMACEUTICALS CORP., Lexington, MA (US); THE INSTITUTE FOR CANCER RESEARCH D/B/A THE RESEARCH INSTITUTE OF FOX CHASE CANCER CENTER, Philadelphia, PA (US)

(72) Inventors: David Proia, Newton, MA (US); Erica Golemis, Oreland, PA (US); Tamina Seeger-Nukpezah, Cologne (DE)

(21) Appl. No.: 14/436,401

(22) PCT Filed: Oct. 18, 2013
(86) PCT No.: PCT/US2013/065725

§ 371 Date: Apr. 16, 2015

Related U.S. Application Data
(60) Provisional application No. 61/716,173, filed on Oct. 19, 2012.

(51) Int. Cl. A61K 31/541; A61K 31/5377; A61K 45/06; A61K 31/496; A61K 31/55; A61K 31/401; A61K 31/4196; A61K 31/4439

(52) U.S. Cl. CPC A61K 31/541; A61K 31/4196; A61K 31/5377; A61K 31/4439; A61K 31/496; A61K 31/55; A61K 31/401; A61K 45/06

(57) ABSTRACT

Provided are methods of treating polycystic kidney disease in a subject in need thereof, by administering to the subject in need thereof, an effective amount of a compound according to formula (I) or a pharmaceutically acceptable salt thereof, wherein the variables in the structural formulae are defined herein. Also provided are methods of treating polycystic kidney disease by a compound of formula (I) in combination with additional therapeutic agents.

### CROSS-REFERENCE TO RELATED PATENTS

This application claims the benefit of priority to U.S. Provisional Patent Application No. 61/716,173 filed on Oct. 19, 2012. The contents of the above application are incorporated herein by reference in their entirety.

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer. Examples of Hsp90 client proteins that have been implicated in the progression of cancer are described below.

Her-2 is a transmembrane tyrosine kinase cell surface growth factor receptor that is expressed in normal epithelial cells. Her2 has an extracellular domain that interacts with extracellular growth factors and an internal tyrosine kinase portion that transmits the external growth signal to the nucleus of the cell. Her2 is overexpressed in a significant proportion of malignancies, such as breast cancer, ovarian cancer, prostate cancer, and gastric cancers, and is typically associated with a poor prognosis.

c-Kit is a membrane receptor protein tyrosine kinase which binds Stem Cell Factor (SCF) to its extraellular domain. c-Kit is involved in the development of melanocytes, mast, germ and hematopoietic cells, and there is evidence that it plays a role in several types of cancer including leukemias, mast cell tumors, small cell lung cancer, testicular cancer, cancers of the gastrointestinal tract and cancers of the central nervous system.

c-Met is a receptor tyrosine kinase that is encoded by the Met protooncogene and transduces the biological effects of hepatocyte growth factor (HGF), which is also referred to as scatter factor (SF). Jiang et al., Crit. Rev. Oncol. Hemtol. 29: 209-248 (1999), the entire teachings of which are incorporated herein by reference. c-Met and HGF are expressed in numerous tissues, although their expression is normally confined predominantly to cells of epithelial and mesenchymal origin, respectively. c-Met and HGF are required for normal mammalian development and have been shown to be important in cell migration, cell proliferation and survival, morphogenic differentiation, and organization of 3-dimensional tubular structures (e.g., renal tubular cells, gland formation, etc.). The c-Met receptor has been shown to be expressed in a number of human cancers. c-Met and its ligand, HGF, have also been shown to be co-expressed at elevated levels in a variety of human cancers (particularly sarcomas). However, because the receptor and ligand are usually expressed by different cell types, c-Met signaling is most commonly regulated by tumor-stroma (tumor-host) interactions. Furthermore, c-Met gene amplification, mutation, and rearrangement have been observed in a subset of human cancers. Families with germine mutations that activate c-Met kinase are prone to multiple kidney tumors as well as tumors in other tissues. Numerous studies have correlated the expression of c-Met and/or HGF/SF with the state of disease progression of different types of cancer (including lung, colon, breast, prostate, liver, pancreas, brain, kidney, ovaries, stomach, skin, and bone cancers). Furthermore, the overexpression of c-Met or HGF have been shown to correlate with poor prognosis and disease outcome in a number of major human cancers including lung, liver, gastric, and breast.

Akt kinase is a serine/threonine kinase which is a downstream effector molecule of phosphoinositide 3-kinase and is involved in protecting the cell from apoptosis. Akt kinase is thought to be involved in the progression of cancer because it stimulates cell proliferation and suppresses apoptosis.

Cdk4/cyclin D complexes are involved in phosphorylation of retinoblastoma protein which is an essential step in progression of a cell through the G1 phase of the cell cycle. Disruption of Hsp90 activity has been shown to decrease the half-life of newly synthesized Cdk4.

Raf-1 is a MAP 3-kinase (MAP3K) which when activated can phosphorylate and activate the serine/threonine specific protein kinases ERK1 and ERK2. Activated ERKs play an important role in the control of gene expression involved in the cell division cycle, apoptosis, cell differentiation and cell migration.

The transforming protein of Rous sarcoma virus, v-src, is a prototype of an oncogene family that induces cellular transformation (i.e., tumorogenesis) by non-regulated kinase activity. Hsp90 has been shown to complex with v-scr and inhibit its degradation.

The BCR-ABL fusion protein associated with chronic myelogenous leukemia and in a subset of patients with acute lymphoblastic leukemia. The fusion protein is a consequence of exchange of genetic material from the long arms of chromosomes 9 and 22 and results in unregulated tyrosine kinase activity. BCR-ABL exists as a complex with Hsp90 and is rapidly degraded when the action of Hsp90 is inhibited.

### SUMMARY OF THE INVENTION

It is now found that certain triazolone Hsp90 inhibitors are surprisingly effective at treating subjects with polycystic kidney disease. In an embodiment, the method described herein utilizes an Hsp90 inhibitor according to formulae (I)-(VII), or a compound in Table 1, or a pharmaceutically acceptable salt thereof, for treating polycystic kidney disease in a subject in need thereof. In an embodiment, the method of treating the subject with polycystic kidney disease includes administering to the subject in need thereof, an effective amount of an Hsp90 inhibitor according to formulae (I)-(VII) or a compound in Table 1, or a pharmaceutically acceptable salt thereof. In an embodiment, the Hsp90 inhibitor is administered as a single agent. In another embodiment, the Hsp90 inhibitor is administered in combination with one or more additional therapeutic agents.

In an embodiment, the method includes the use of an Hsp90 inhibitor according to formulae (I)-(VII) or a compound in Table 1or a pharmaceutically acceptable salt thereof, for the manufacture of a medicament for treating polycystic kidney disease in a subject in need thereof. In another embodiment, the method includes the use of an Hsp90 inhibitor according to formulae (I)-(VII) or a compound in Table 1, or a pharmaceutically acceptable salt thereof, for the manufacture of a medicament for treating polycystic kidney disease in a subject in need thereof, in combination with one or more additional therapeutic agents.

In an embodiment, the method includes treating polycystic kidney disease in a subject by administering to the subject an effective amount of an Hsp90 compound according to formulae (I)-(VII) or a compound in Table 1, or a pharmaceutically acceptable salt thereof. In an embodiment, the method treating polycystic kidney disease may include the administration of one or more therapeutic agents in addition to an Hsp90 compound according to formulae (I)-(VII) or a compound in Table 1.

### BRIEF DESCRIPTION OF THE DRAWINGS

The foregoing and other objects, features and advantages of the invention will be apparent from the following more particular description of some embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows that compound 111 inhibited cyst formation, kidney growth in Pkd1^{-/-} mice. PKD1 null mice were treated with vehicle or 75 mg/kg of compound 111 for 5 months. The representative MRI pictures of FIG. 1 are from 1, 4, 5 and 6 month of age and corresponding H&E-stained kidney sections.

FIG. 2 shows the results of kidney volumes and cyst volume at 6 months for the PKD1 null mice treated with vehicle or 75 mg/kg of compound 111. Error bar represents standard error; *p<0.05; **p,0.005.

FIG. 3 shows the results of kidney weighting, blood urea nitrogen (BUN) cystic index for the PKD1 null mice treated with vehicle or 75 mg/kg of compound 111. Error bar represents standard error; *p<0.05; **p,0.005.

FIG. 4 shows that compound 111 reduced cystic burden in a mouse model of PKD. PKD1 null mice were treated with vehicle, 50 mg/kg, or 100 mg/kg of compound 111 for 10 weeks. The drug was administered by tail vein injection 1×/week. The representative MRI pictures of FIG. 4 are from 0, 5, and 10 weeks after the treatment and corresponding H&E-stained kidney sections.

FIG. 5 shows the results of kidney volumes and cyst volume at 10 weeks for the PKD1 null mice treated with vehicle, 50 mg/kg, or 100 mg/kg of compound 111 for 10 weeks. Error bar represents standard error; *p<0.05; **p,0.005.

FIG. 6 shows the results of kidney weighting, blood urea nitrogen (BUN) cystic index for the PKD1 null mice treated with vehicle or 50 mg/kg, or 100 mg/kg of compound 111 for 10 weeks. Error bar represents standard error; *p<0.05; **p,0.005.

### DETAILED DESCRIPTION OF THE INVENTION

### Definitions

Unless otherwise specified, the below terms used herein are defined as follows:

As used herein, the term “alkyl” means a saturated or unsaturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds described herein may be optionally substituted with one or more substituents. Examples of unsaturated alkyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkyl groups included in compounds described herein may be optionally substituted with one or more substituents.

As used herein, the term “cycloalkyl” means a saturated or unsaturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, cyclohexenyl, cyclooctenyl, cyclohexynyl, and the like. Cycloalkyl groups included in the compounds described herein may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be saturated or unsaturated, and may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker. Alkoxy groups included in compounds described herein may be optionally substituted with one or more substituents.

As used herein, a “haloalkoxy” is a haloalkyl group which is attached to another moiety via an oxygen linker.

As used herein, the term an “aromatic ring” or “aryl” means a mono- or polycyclic hydrocarbon, containing from 6 to 15 carbon atoms, in which at least one ring is aromatic. Examples of suitable aryl groups include phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds described herein may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds described herein may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds described herein may be optionally substituted with one or more substituents. Only stable isomers of such substituted heterocyclic groups are contemplated in this definition.

As used herein, the term “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, an isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds described herein may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds described herein may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound described herein without significantly adversely affecting the reactivity or biological activity of the compound described herein. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, —NR³³C(O)R³¹, —NR³³C(S)R³¹, —NR³³C(NR³²)R³¹, halo, —OR³³, cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³⁰C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C(NR³²)OR³¹, —S(O)ₖR³³, —OS(O)ₖR³³, —NR³³S(O)ₖR³³, —S(O)ₖNR²⁸R²⁹, —OS(O)ₖNR²⁸R²⁹, —NR³³S(O)ₖNR²⁸R²⁹, guanidino, —C(O)SR³¹, —C(S)SR³¹, —C(NR³²)SR³¹, —OC(O)OR³¹, —OC(S)OR³¹, —OC(NR³²)OR³¹, —SC(O)R³³, —SC(O)OR³¹, —SC(NR³²)OR³¹, —SC(S)R³³, —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₖOR³¹, —S(O)ₖOR³¹, —NR³⁰S(O)ₖOR³¹, —SS(O)ₖR³³, —SS(O)ₖOR³¹, —SS(O)ₖNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³². Each R²⁸ and R²⁹ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹ is optionally and independently substituted. Each R³⁰, R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³⁰ or R³¹ or R³³ is optionally and independently unsubstituted. Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₖR³³, or —S(O)ₖNR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted. The variable k is 0, 1 or 2. In some embodiments, suitable substituents include C1-C4 alkyl, C1-C4 haloalkyl, C1-C4 alkoxy, C1-C4 haloalkoxy, C1-C4 hydroxyalkyl, halo, or hydroxyl.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

Her2 is a transmembrane tyrosine kinase cell surface growth factor receptor that is expressed in normal epithelial cells. Her2 has an extracellular domain that interacts with extracellular growth factors and an internal tyrosine kinase portion that transmits the external growth signal transduction pathways leading to cell growth and differentiation. Her2 is overexpressed in a significant proportion of malignancies, such as breast cancer, ovarian cancer, prostate cancer and gastric cancers, and is typically associated with a poor prognosis. It is encoded within the genome by HER2/neu, a known proto-oncogene. HER2 is thought to be an orphan receptor, with none of the EGF family of ligands able to activate it. However, ErbB receptors dimerise on ligand binding, and HER2 is the preferential dimerisation partner of other members of the ErbB family. The HER2 gene is a proto-oncogene located at the long arm of human chromosome 17(17q21-q22). HER2/neu (also known as ErbB-2) stands for “Human Epidermal growth factor Receptor 2” and is a protein giving higher aggressiveness in breast cancers. It is a member of the ErbB protein family, more commonly known as the epidermal growth factor receptor family. HER2/neu has also been designated as CD340 (cluster of differentiation 340) and p185. Approximately 15-20 percent of breast cancers have an amplification of the HER2/neu gene or overexpression of its protein product. Overexpression of this receptor in breast cancer is associated with increased disease recurrence and worse prognosis.

The Anaplastic Lymphoma Kinase (ALK) tyrosine kinase receptor is an enzyme that in humans is encoded by the ALK gene. The 2;5 chromosomal translocation is frequently associated with anaplastic large cell lymphomas (ALCLs). The translocation creates a fusion gene consisting of the ALK (anaplastic lymphoma kinase) gene and the nucleophosmin (NPM) gene: the 3′ half of ALK, derived from chromosome 2, is fused to the 5′ portion of NPM from chromosome 5. The product of the NPM-ALK fusion gene is oncogenic. Other possible translocations of the ALK gene, such as the elm4 translocation, are also implicated in cancer.

B-Raf proto-oncogene serine/threonine-protein kinase (B-RAF), also known as V-raf murine sarcoma viral oncogene homolog B1, is a protein that in humans is encoded by the BRAF gene. The B-RAF protein is involved in sending signals in cells and in cell growth. The BRAF gene may be mutated, and the B-RAF protein altered, as an inherited mutation which causes birth defects, or as an acquired mutation (oncogene) in adults which causes cancer. Acquired mutations in this gene have also been found in cancers, including non-Hodgkin lymphoma, colorectal cancer, malignant melanoma, papillary thyroid carcinoma, non-small cell lung carcinoma, and adenocarcinoma of lung. More than 30 mutations of the BRAF gene associated with human cancers have been identified. The frequency of BRAF mutations varies widely in human cancers from more than 80% in melanomas, to as little as 0-18% in other tumors, such as 1-3% in lung cancers and 5% in colorectal cancer. In 90% of the cases, a Glu for Val substitution at residue 599 (now referred to as V600E) in the activation segment has been found in human cancers. This mutation has been widely observed in papillary thyroid carcinoma, colorectal cancer and melanomas. Depending on the type of mutation the kinase activity towards MEK may also vary. In the same paper it has been reported that most of the mutants stimulate enhanced B-RAF kinase activity toward MEK. However, a few mutants act through a different mechanism because although their activity toward MEK is reduced, they adopt a conformation that activates wild-type C-RAF, which then signals to ERK.

KRAS is a protein which in humans is encoded by the KRAS gene. Like other members of the Ras family, the KRAS protein is a GTPase and is an early player in many signal transduction pathways. KRAS is usually tethered to cell membranes because of the presence of an isoprenyl group on its C-terminus. When mutated, KRAS is an oncogene. The protein product of the normal KRAS gene performs an essential function in normal tissue signaling, and the mutation of a KRAS gene is an essential step in the development of many cancers. KRAS acts as a molecular on/off switch, and once it is turned on it recruits and activates proteins necessary for the propagation of growth factor and other receptors' signal, such as c-Raf and PI 3-kinase.

Phosphoinositide 3-kinases (PI 3-kinases or PI3Ks) are a family of enzymes involved in cellular functions such as cell growth, proliferation, differentiation, motility, survival and intracellular trafficking, which in turn are involved in cancer. PI3Ks are a family of related intracellular signal transducer enzymes capable of phosphorylating the 3 position hydroxyl group of the inositol ring of phosphatidylinositol (PtdIns). They are also known as phosphatidylinositol-3-kinases. The pathway, with oncogene PIK3CA and tumor suppressor PTEN (gene) is implicated in insensitivity of cancer tumors to insulin and IGF1, in calorie restriction. PI 3-kinases have been linked to an extraordinarily diverse group of cellular functions, including cell growth, proliferation, differentiation, motility, survival and intracellular trafficking. Many of these functions relate to the ability of class I PI 3-kinases to activate protein kinase B (PKB, aka Akt). The class IA PI 3-kinase p110α is mutated in many cancers. Many of these mutations cause the kinase to be more active. The PtdIns (3,4,5)P₃ phosphatase PTEN that antagonises PI 3-kinase signaling is absent from many tumors. Hence, PI 3-kinase activity contributes significantly to cellular transformation and the development of cancer.

AKT protein family, which members are also called protein kinases B (PKB) plays an important role in mammalian cellular signaling. Akt kinase is a serine/threonine kinase which is a downstream effector molecule of phosphoinositide 3-kinase and is involved in protecting a cell from apoptosis. Akt kinase is thought to be involved in the progression of cancer because it stimulates cell proliferation and suppresses apoptosis. Akt1 is involved in cellular survival pathways, by inhibiting apoptotic processes. Akt1 is also able to induce protein synthesis pathways, and is therefore a key signaling protein in the cellular pathways that lead to skeletal muscle hypertrophy, and general tissue growth. Since it can block apoptosis, and thereby promote cell survival, Akt1 has been implicated as a major factor in many types of cancer. Akt is known to play a role in the cell cycle. Under various circumstances, activation of Akt was shown to overcome cell cycle arrest in G1 and G2 phases. Moreover, activated Akt may enable proliferation and survival of cells that have sustained a potentially mutagenic impact and, therefore, may contribute to acquisition of mutations in other genes.

Cdk4/cyclin D complexes are involved in phosphorylation of the retinoblastoma protein, which is an essential step in progression of a cell through the G1 phase of the cell cycle. Disruption of Hsp90 activity has been shown to decrease the half life of newly synthesized Cdk4.

Raf-1 is a MAP 3-kinase (MAP3K) which, when activated, can phosphorylate and activate the serine/threonine specific protein kinases ERK1 and ERK2. Activated ERKs play an important role in the control of gene expression involved in the cell division cycle, apoptosis, cell differentiation and cell migration.

The transforming protein of the Rous sarcoma virus, v-src, is a prototype of an oncogene family that induces cellular transformation (i.e., tumorogenesis) by non-regulated kinase activity. Hsp90 has been shown to complex with v-scr and inhibit its degradation.

p53 is a tumor suppressor protein that causes cell cycle arrest and apoptosis. Mutation of the p53 gene is found in about half of all human cancers, making it one of the most common genetic alterations found in cancerous cells. In addition, the p53 mutation is associated with a poor prognosis. Wild-type p53 has been shown to interact with Hsp90, but mutated p53 forms a more stable association with Hsp90 than wild-type p53 as a result of its misfolded conformation. A stronger interaction with Hsp90 protects the mutated protein from normal proteolytic degradation and prolongs its half-life. In a cell that is heterozygous for mutated and wild-type p53, inhibition of the stabilizing effect of Hsp90 causes mutant p53 to be degraded and restores the normal transcriptional activity of wild-type p53.

There are two classes of protein kinases (PKs): protein tyrosine kinases (PTKs), which catalyze the phosphorylation of tyrosine kinase residues, and the serine-threonine kinases (STKs), which catalyze the phosphorylation of serine or threonine residues. Growth factor receptors with PTK activity are known as receptor tyrosine kinases. Receptor tyrosine kinases are a family of tightly regulated enzymes, and the aberrant activation of various members of the family is one of the hallmarks of cancer. The receptor tyrosine kinase family can be divided into subgroups that have similar structural organization and sequence similarity within the kinase domain.

The members of the type III group of receptor tyrosine kinases include platelet-derived growth factor receptors (PDGF receptors alpha and beta), colony-stimulating factor receptor (CSF-1R, c-Fms), Fms-like tyrosine kinase (FLT3), and stem cell factor receptor (c-Kit). FLT3 is primarily expressed on immature hematopoietic progenitors and regulates their proliferation and survival.

The FLT3-ITD mutation is also present in about 3% of cases of adult myelodysplastic syndrome and some cases of acute lymphocytic leukemia (ALL). Advani, Current Pharmaceutical Design (2005), 11:3449-3457. FLT3 has been shown to be a client protein of Hsp90, and 17AAG, a benzoquinone ansamycin antibiotic that inhibits Hsp90 activity, has been shown to disrupt the association of FLT3 with Hsp90. The growth of leukemia cells that express either wild type FLT3 or FLT3-ITD mutations was found to be inhibited by treatment with 17AAG. Yao, et al., Clinical Cancer Research (2003), 9:4483-4493.

c-Kit is a membrane type III receptor protein tyrosine kinase which binds Stem Cell Factor (SCF) to its extraellular domain. c-Kit has tyrosine kinase activity and is required for normal hematopoiesis. However, mutations in c-Kit can result in ligand-independent tyrosine kinase activity, autophosphorylation and uncontrolled cell proliferation. Aberrant expression and/or activation of c-Kit has been implicated in a variety of pathologic states. For example, there is evidence of a contribution of c-Kit to neoplastic pathology, including its association with leukemias and mast cell tumors, small cell lung cancer, testicular cancer and some cancers of the gastrointestinal tract and central nervous system. In addition, c-Kit has been implicated in carcinogenesis of the female genital tract, sarcomas of neuroectodermal origin, and Schwann cell neoplasia associated with neurofibromatosis. Yang et al., J Clin Invest. (2003), 112:1851-1861; Viskochil, J Clin Invest. (2003), 112:1791-1793. c-Kit has been shown to be a client protein of Hsp90, and Hsp90 inhibitor 17AAG has been shown to induce apoptosis in Kasumi-1 cells, an acute myeloid leukemia cell line that harbors a mutation in c-Kit.

c-Met is a receptor tyrosine kinase that is encoded by the Met protooncogene and transduces the biological effects of hepatocyte growth factor (HGF), which is also referred to as scatter factor (SF). Jiang, et al., Crit. Rev. Oncol. Hemtol. (1999), 29: 209-248. c-Met and HGF are expressed in numerous tissues, although their expression is normally predominantly confined to cells of epithelial and mesenchymal origin, respectively. c-Met and HGF are required for normal mammalian development and have been shown to be important in cell migration, cell proliferation, cell survival, morphogenic differentiation and the organization of 3-dimensional tubular structures (e.g., renal tubular cells, gland formation, etc.). The c-Met receptor has been shown to be expressed in a number of human cancers. c-Met and its ligand, HGF, have also been shown to be co-expressed at elevated levels in a variety of human cancers, particularly sarcomas. However, because the receptor and ligand are usually expressed by different cell types, c-Met signaling is most commonly regulated by tumor-stroma (tumor-host) interactions. Furthermore, c-Met gene amplification, mutation and rearrangement have been observed in a subset of human cancers. Families with germine mutations that activate c-Met kinase are prone to multiple kidney tumors, as well as tumors in other tissues. Numerous studies have correlated the expression of c-Met and/or HGF/SF with the state of disease progression of different types of cancer, including lung, colon, breast, prostate, liver, pancreas, brain, kidney, ovarian, stomach, skin and bone cancers. Furthermore, the overexpression of c-Met or HGF have been shown to correlate with poor prognosis and disease outcome in a number of major human cancers including lung, liver, gastric and breast.

BCR-ABL is an oncoprotein with tyrosine kinase activity that has been associated with chronic myelogenous leukemia (CML), acute lymphocytic leukemia (ALL) in a subset of patients and acute myelogenous leukemia (AML) in a subset of patients. In fact, the BCR-ABL oncogene has been found in at least 90-95% of patients with CML, about 20% of adults with ALL, about 5% of children with ALL and in about 2% of adults with AML. The BCR-ABL oncoprotein is generated by the translocation of gene sequences from the c-ABL protein tyrosine kinase on chromosome 9 into the BCR sequences on chromosome 22, producing the Philadelphia chromosome. The BCR-ABL gene has been shown to produce at least three alternative chimeric proteins, p230 BCR-ABL, p210 BCR-ABL and p190 BCR-ABL, which have unregulated tyrosine kinase activity. The p210 BCR-ABL fusion protein is most often associated with CML, while the p190 BCR-ABL fusion protein is most often associated with ALL. BCR-ABL has also been associated with a variety of additional hematological malignancies including granulocytic hyperplasia, myelomonocytic leukemia, lymphomas and erythroid leukemia. BCR-ABL fusion proteins exist as complexes with Hsp90 and are rapidly degraded when the action of Hsp90 is inhibited. It has been shown that geldanamycin, a benzoquinone ansamycin antibiotic that disrupts the association of BCR-ABL with Hsp90, results in proteasomal degradation of BCR-ABL and induces apoptosis in BCR-ABL leukemia cells.

Epidermal Growth Factor Receptor (EGFR) is a member of the type 1 subgroup of receptor tyrosine kinase family of growth factor receptors which play critical roles in cellular growth, differentiation and survival. Activation of these receptors typically occurs via specific ligand binding which results in hetero- or homodimerization between receptor family members, with subsequent autophosphorylation of the tyrosine kinase domain. Specific ligands which bind to EGFR include epidermal growth factor (EGF), transforming growth factor α (TGFα), amphiregulin and some viral growth factors. Activation of EGFR triggers a cascade of intracellular signaling pathways involved in both cellular proliferation (the ras/raf/MAP kinase pathway) and survival (the PI3 kinase/Akt pathway). Members of this family, including EGFR and HER2, have been directly implicated in cellular transformation.

A number of human malignancies are associated with aberrant or overexpression of EGFR and/or overexpression of its specific ligands. Gullick, Br. Med. Bull. (1991), 47:87-98; Modijtahedi & Dean, Int. J. Oncol. (1994), 4:277-96; Salomon, et al., Crit. Rev. Oncol. Hematol. (1995), 19:183-232. Aberrant or overexpression of EGFR has been associated with an adverse prognosis in a number of human cancers, including head and neck, breast, colon, prostate, lung (e.g., NSCLC, adenocarcinoma and squamous lung cancer), ovarian, gastrointestinal cancers (gastric, colon, pancreatic), renal cell cancer, bladder cancer, glioma, gynecological carcinomas and prostate cancer. In some instances, overexpression of tumor EGFR has been correlated with both chemoresistance and a poor prognosis. Lei, et al., Anti-cancer Res. (1999), 19:221-28; Veale, et al., Br. J. Cancer (1993); 68:162-65. Mutations in EGFR are associated with many types of cancer as well. For example, EGFR mutations are highly prevalent in non-mucinous BAC patients. Finberg, et al., J. Mol. Diagnostics (2007) 9(3):320-26.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term “about”.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In another embodiment, the subject is a human.

Unless indicated otherwise, the compounds described herein containing reactive functional groups, such as, for example, carboxy, hydroxy, thiol and amino moieties, also include corresponding protected derivatives thereof. “Protected derivatives” are those compounds in which a reactive site or sites are blocked with one ore more protecting groups. Examples of suitable protecting groups for hydroxyl groups include benzyl, methoxymethyl, allyl, trimethylsilyl, tert-butyldimethylsilyl, acetate, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like. Other suitable protecting groups are well known to those of ordinary skill in the art and include those found in T. W. GREENE, PROTECTING GROUPS IN ORGANIC SYNTHESIS, (John Wiley & Sons, Inc., 1981).

As used herein, the term “compound(s) described herein” or similar terms refers to a compound of formulae (I)-(VII) or a compound in Table 1, or a pharmaceutically acceptable salt thereof. Also included in the scope of the embodiments are a solvate, clathrate, hydrate, polymorph, prodrug, or protected derivative of a compound of formulae (I)-(VII), or a compound in Table 1.

The compounds described herein may contain one or more chiral centers and/or double bonds and, therefore, exist as stereoisomers, such as double-bond isomers (i.e., geometric isomers), enantiomers or diastereomers. Each chemical structure shown herein, including the compounds described herein, encompass all of the corresponding compound' enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds described herein are preferred.

When a disclosed compound is named or depicted by structure, it is to be understood that solvates (e.g., hydrates) of the compound or a pharmaceutically acceptable salt thereof is also included. “Solvates” refer to crystalline forms wherein solvent molecules are incorporated into the crystal lattice during crystallization. Solvates may include water or nonaqueous solvents such as ethanol, isopropanol, DMSO, acetic acid, ethanolamine and ethyl acetate. When water is the solvent molecule incorporated into the crystal lattice of a solvate, it is typically referred to as a “hydrate”. Hydrates include stoichiometric hydrates as well as compositions containing variable amounts of water.

When a disclosed compound is named or depicted by structure, it is to be understood that the compound, including solvates thereof, may exist in crystalline forms, non-crystalline forms or a mixture thereof. The compounds or solvates may also exhibit polymorphism (i.e., the capacity to occur in different crystalline forms). These different crystalline forms are typically known as “polymorphs.” It is to be understood that when named or depicted by structure, the disclosed compounds and solvates (e.g., hydrates) also include all polymorphs thereof. Polymorphs have the same chemical composition but differ in packing, geometrical arrangement and other descriptive properties of the crystalline solid state. Polymorphs, therefore, may have different physical properties such as shape, density, hardness, deformability, stability and dissolution properties. Polymorphs typically exhibit different melting points, IR spectra and X-ray powder diffraction patterns, which may be used for identification. One of ordinary skill in the art will appreciate that different polymorphs may be produced, for example, by changing or adjusting the conditions used in crystallizing the compound. For example, changes in temperature, pressure or solvent may result in different polymorphs. In addition, one polymorph may spontaneously convert to another polymorph under certain conditions.

When a disclosed compound is named or depicted by structure, it is to be understood that clathrates (“inclusion compounds”) of the compound or its pharmaceutically acceptable salt, solvate or polymorph, are also included. “Clathrate” means a compound described herein, or a salt thereof, in the form of a crystal lattice that contains spaces (e.g., channels) that have a guest molecule trapped within (e.g., a solvent or water).

As used herein, and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated herein include analogs or derivatives of compounds of formulae (I)-(VII) or a compound in Table 1 that comprise biohydrolyzable moieties such as biohydrolyzable amides, biohydrolyzable esters, biohydrolyzable carbamates, biohydrolyzable carbonates, biohydrolyzable ureides and phosphate analogues. Prodrugs can typically be prepared using well-known methods, such as those described by BURGER′S MEDICINAL CHEMISTRY AND DRUG DISCOVERY, (Manfred E. Wolff Ed., 5th ed. (1995)) 172-178, 949-982.

As used herein, “Hsp90” includes each member of the family of heat shock proteins having a mass of about 90-kiloDaltons. For example, in humans the highly conserved Hsp90 family includes the cytosolic Hsp90α and Hsp90β isoforms, as well as GRP94, which is found in the endoplasmic reticulum, and HSP75/TRAP1, which is found in the mitochondrial matrix. Some exemplary Hsp90 inhibitors include geldanamycin derivatives, e.g., a benzoquinone or hygroquinone ansamycin HSP90 inhibitor such as IPI-493 (CAS No. 64202-81-9) and/or IPI-504 (CAS No. 857402-63-2); 17-AAG CAS No. 75747-14-7), BIIB-021 (CNF-2024, CAS No. 848695-25-0), BUB-028, AUY-922 (also known as VER-49009, CAS No. 747412-49-3), SNX-5422 (CAS No. 908115-27-5), AT-13387 (CAS No. 912999-49-6), XL-888, MPC-3100, CU-0305, 17-DMAG (CAS No. 467214-21-7), CNF-1010 (CAS No. 946090-39-7), Macbecin (e.g., Macbecin I (CAS No. 73341-72-7), Macbecin II (CAS No. 73341-73-8)), CCT-018159 (CAS No. 171009-07-7), CCT-129397 (CAS No. 940289-57-6), PU-H71 (CAS No. 873436-91-0), or PF-04928473 (SNX-2112, CAS No. 945626-71-1), 1,2,4-triazole derivatives such as ganetespib, and compound 111 or Cpd 111 (5-(2,4-dihydroxy-5-isopropylphenyl)-N-(2-morpholinoethyl)-4-(4-(morpholinomethyl)phenyl)-4H-1,2,4-triazole-3-carboxamide).

Autosomal dominant polycystic kidney disease (ADPKD) is a common genetically inherited disorder that poses a very significant public health burden. ADPKD affects between 1:400 and 1:1000 individuals worldwide. Symptoms typically manifest at middle age, with kidney function increasingly impaired as normal tissue becomes replaced by thousands of abnormally proliferating cysts. The age of onset and severity of the disease are influenced by the nature of the mutation in PKD1 or PKD2, as well as by gene modifiers, although relatively few of the latter have been well defined. There is currently no effective treatment for ADPKD, with patients typically progressing towards a requirement for renal transplantation or dialysis. ADPKD arises from mutations in the genes PKD1 (˜85% of cases, based on clinical diagnosis) or PKD2 (˜15% of cases, by the same criterion), which heterodimerize at the plasma membrane of normal renal cells to transmit numerous signals that regulate cell growth. Formation of cysts is marked by multiple phenotypic changes in the cells lining renal tubules. These include loss of epithelial planar cell polarity, dysfunction in centrosomes, increased proliferation and apoptosis, changes of basal cell attachments and the extracellular matrix (ECM), altered intracellular calcium levels, and deregulation of multiple signal transduction pathways. These pleiotropic changes reflect the complex cellular action of PC1 and PC2, products of the PKD1 and PKD2 genes, which is typically reduced or lost following mutation in ADPKD. Kidney cells with mutated PKD genes are characterized by enhanced activation of many pro-proliferative signaling proteins that include HER2, mTOR, STAT3, SRC, AKT, ERK1/2, RAF, mTOR, S6, and others. Therapeutic strategies currently or previously under evaluation for use in ADPKD include inhibitors of some of these proteins, including SKI-606 (targeting SRC), sirolimus and folate-conjugated rapamycin (targeting mTOR), and others.

PC1 and PC2 are both large trans-membrane proteins with numerous interacting partners, which allow them to influence multiple cell growth regulatory pathways. PC2 is a transient receptor potential (TRP) calcium channel; PC1 is distant relative of a TRP channel, but with an extended extracellular N-terminal domain containing multiple interactive motifs. In normal renal cells, a PC1-PC2 heterodimer localizes to the primary cell cilium, which protrudes into the lumen and senses fluid flow, then communicates growth-restrictive signals to the cell. A number of studies have shown that genetic lesions that cause loss of cilia are sufficient to generate loss of planar cell polarity and induce renal cysts, indirectly and in combination with other studies indicating that ability to localize to the cilium is important for normal PC1-PC2 function. Other important PC1 functions include association with E-cadherin complexes, regulating the level of E-cadherin at cell-cell junctions in response to intracellular calcium levels, and association with focal adhesion components, to control cell-ECM interactions. Further, following cleavage induced by abnormal luminal fluid flow, the isolated C-terminus of the protein can translocate to the nucleus and regulate β-catenin-dependent transcription of Myc, cyclin D, and other genes affecting cell cycle. Direct and indirect interactions between PC2 and proteins such as ID2, eIF2α, and mTOR limit cellular growth by affecting the processes of transcription and translation. PC2 also localizes to multiple cellular compartments beyond the cilium, including the plasma membrane, endoplasmic reticulum (ER), centrosome, and mitotic spindle. Many PC2 interacting proteins trigger its calcium channel activity at specific intracellular locales in response to specific physiological stimuli. These range from activation of the epidermal growth factor receptor (EGFR) and its downstream effectors RAF/MEK/ERK, to triggering of Diaphanous signaling on the mitotic apparatus during cell division.

It is known that a certain molecule known as cAMP is involved in the enlargement of kidney cysts in PKD kidneys. Studies on rodents have shown that a molecule, called vasopressin, increases levels of cAMP in the body. When mice with PKD were given a chemical that blocks vasopressin, there was an impressive decrease in kidney size and some preservation of kidney function. Similarly, when studied mice consumed excessive amounts of water (which decreases levels of vasopressin), a similar result was seen. It has therefore been suggested that consuming large amounts of water may possibly assist in the treatment of early stage PKD.

As humans do not always mimic rodents in clinical trials, it is currently not yet certain whether vasopressin inhibitors, such as water, will have corresponding results in humans, or what negative effects excessive water intake may have on the kidneys of individuals with PKD. Clinical trials are currently underway in this field.

It has also been suggested that treatment with medications inhibiting vasopressin may assist in the management of PKD and reduce the speed at which kidney cysts form and grow, delaying the onset of end stage renal failure.

A definite diagnosis of ADPKD relies on imaging or molecular genetic testing. The sensitivity of testing is nearly 100% for all patients with ADPKD who are age 30 years or older and for younger patients with PKD1 mutations. These criteria are only 67% sensitive for patients with PKD2 mutations who are younger than age 30 years. Large echogenic kidneys without distinct macroscopic cysts in an infant/child at 50% risk for ADPKD are diagnostic. In the absence of a family history of ADPKD, the presence of bilateral renal enlargement and cysts, with or without the presence of hepatic cysts, and the absence of other manifestations suggestive of a different renal cystic disease provide presumptive, but not definite, evidence for the diagnosis.

Molecular genetic testing by linkage analysis or direct mutation screening is available clinically; however, genetic heterogeneity is a significant complication to molecular genetic testing. Sometimes a relatively large number of affected family members need to be tested in order to establish which one of the two possible genes is responsible within each family. In the research setting, mutation detection rates of 50-75% have been obtained for PKD1 and ˜75% for PKD2. Clinical testing of the PKD1 and PKD2 genes by direct sequence analysis is now available, with a detection rate for disease-causing mutations of 50-70%.

As used herein, a “proliferative disorder” or a “hyperproliferative disorder,” and other equivalent terms, means a disease or medical condition involving pathological growth of cells. Proliferative disorders include cancer, smooth muscle cell proliferation, systemic sclerosis, cirrhosis of the liver, adult respiratory distress syndrome, idiopathic cardiomyopathy, lupus erythematosus, retinopathy, (e.g., diabetic retinopathy or other retinopathies), cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis and desmoid tumors. Non-cancerous proliferative disorders also include hyperproliferation of cells in the skin such as psoriasis and its varied clinical forms, Reiter's syndrome, pityriasis rubra pilaris, hyperproliferative variants of disorders of keratinization (e.g., actinic keratosis, senile keratosis), scleroderma, and the like. In one embodiment, the proliferative disorder is a myeloproliferative disorder. In one aspect, the myeloproliferative disorder is polycythemia vera, idiopathic myelofirbrosis, myelodysplastic syndrome, psoriasis or essential thrombocythemia. In one embodiment, the proliferative disorder expresses JAK2V617F mutation of JAK2. In an aspect of this embodiment, the proliferative disorder is polycythemia vera, idiopathic myelofirbrosis, or essential thrombocythemia. In one aspect, the proliferative disorder is polycythemia vera.

As used herein, the term “pharmaceutically acceptable salt” refers to a salt prepared from a compound of formulae (I), (II), (III), (IV), (V), (VI, or (VII) or a compound in Table 1 having an acidic functional group, such as a carboxylic acid functional group, and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N, N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of formulae (I)-(VII) or a compound in Table 1 having a basic functional group, such as an amine functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

As used herein, the term “pharmaceutically acceptable solvate,” is a solvate formed from the association of one or more pharmaceutically acceptable solvent molecules to one of the compounds of formulae (I)-(VII) or a compound in Table 1. The term “solvate” includes hydrates, e.g., hemihydrate, monohydrate, dihydrate, trihydrate, tetrahydrate, and the like.

A pharmaceutically acceptable carrier may contain inert ingredients which do not unduly inhibit the biological activity of the compound(s) described herein. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON′S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. In one embodiment of the invention, the disease or disorder is a proliferative disorder. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound described herein being used. In cases where no amount is expressly noted, an effective amount should be assumed. Non-limiting examples of an effective amount of a compound described herein are provided herein below. In a specific embodiment, the method includes treating, managing, or ameliorating a disease or disorder, e.g. a proliferative disorder, or one or more symptoms thereof, comprising administering to a subject in need thereof a dose of the Hsp90 inhibitor at least 150 μg/kg, at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, once every 2 days, once every 3 days, once every 4 days, once every 5 days, once every 6 days, once every 7 days, once every 8 days, once every 10 days, once every two weeks, once every three weeks, or once a month.

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation. In another embodiment, the terms “treat”, “treating” and “treatment” also encompass the administration of a compound described herein as a prophylactic measure to patients with a predisposition (genetic or environmental) to any disease or disorder described herein.

As used herein, the terms “therapeutic agent” and “therapeutic agents” refer to any agent(s) that can be used in the treatment of a disease or disorder, e.g. a proliferative disorder, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., a proliferative disorder, or one or more symptoms thereof.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., a proliferative disorder. The ability to utilize lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapeutic agent. The use of the term “in combination” does not restrict the order in which the therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer. In one embodiment, the Hsp90 inhibitor and the one or more additional therapeutic agents are dosed on independent schedules. In another embodiment, the Hsp90 inhibitor and the one or more additional therapeutic agents are dosed on approximately the same schedule. In another embodiment, the Hsp90 inhibitor and the one or more additional therapeutic agents are dosed concurrently or sequentially on the same day. In another embodiment, the Hsp90 inhibitor and the one or more additional therapeutic agents are dosed sequentially on different days.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

A used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a “racemic mixture” means about 50% of one enantiomer and about 50% of is corresponding enantiomer of the molecule. The combination encompasses all enantiomerically-pure, enantiomerically-enriched, diastereomerically pure, diastereomerically enriched, and racemic mixtures of the compounds described herein. Enantiomeric and diastereomeric mixtures can be resolved into their component enantiomers or diastereomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

The compounds described herein are defined by their chemical structures and/or chemical names. Where a compound is referred to by both a chemical structure and a chemical name, and the chemical structure and the chemical name conflict, the chemical structure is determinative of the compound's identity.

When administered to a subject (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomeric or enantiomeric pure isolate.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

In one aspect, the method includes treating polycystic kidney disease in a subject in need thereof, comprising administering an effective amount of an Hsp90 inhibitory compound shown in Table 1, or according to formula (I) as set forth below:

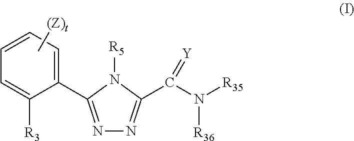

wherein:

Y is O or S;

R₃ is —OH, —SH, —NR₇H, —OR₂₆, —SR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;

R₅ is —H, —X₂₀R₅₀, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₇ and R₈, for each occurrence, is independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₂₆ is a lower alkyl;

R₃₅ and R₃₆, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, or R₃₅ and R₃₆, together with N to which they are attached form a 5 to 7 membered heterocyclic ring;

R₅₀ is an optionally substituted aryl or an optionally substituted heteroaryl;

X₂₀ is a C₁-C₄ alkyl, NR₇, C(O), C(S), C(NR₈), or S(O)ₚ;

Z is a substituent, which is independently —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, 'NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

t is 0, 1, 2, 3, or 4;

p, for each occurrence, is independently, 1 or 2;

or a pharmaceutically acceptable salt thereof.

In one aspect, the method includes treating polycystic kidney disease in a subject in need thereof, comprising administering an effective amount of an Hsp90 inhibitory compound shown in Table 1, or according to formula (II) as set forth below:

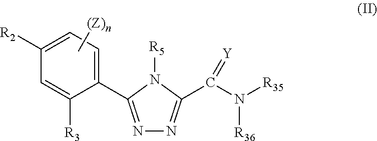

wherein:

R₂ is —OH, —SH, —NR₇H, —OR₂₆, —SR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)NR₇)₂, or —SP(O)(OR₇)₂; and

n is 0, 1, 2, or 3; or a pharmaceutically acceptable salt, solvate, clathrate or a prodrug thereof.

In one aspect, the method includes treating polycystic kidney disease in a subject in need thereof, comprising administering an effective amount of an Hsp90 inhibitory compound shown in Table 1, or according to formula (III) as set forth below:

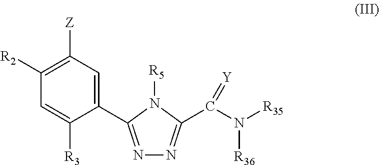

or a pharmaceutically acceptable salt thereof.

In one aspect, the method includes treating polycystic kidney disease in a subject in need thereof, comprising administering an effective amount of an Hsp90 inhibitory compound shown in Table 1, or according to formula (IV) as set forth below:

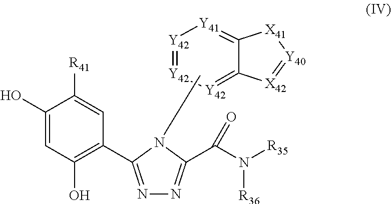

wherein:

X₄₁ is O, S, or NR₄₂;

X₄₂ is CR₄₄ or N;

Y₄₀ is N or CR₄₃;

Y₄₁ is N or CR₄₅;

Y₄₂, for each occurrence, is independently N, C or CR₄₆;

R₄₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;

R₄₂ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;

R₄₃ and R₄₄ are, independently, —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;

R₄₅ is —H, —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁; and

R₄₆, for each occurrence, is independently, selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁;

or a pharmaceutically acceptable salt thereof.

In one aspect, the method includes treating polycystic kidney disease in a subject in need thereof, comprising administering an effective amount of an Hsp90 inhibitory compound shown in Table 1, or according to formula (V) as set forth below:

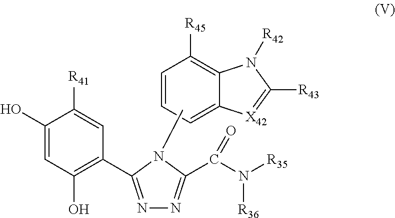

or a pharmaceutically acceptable salt thereof.

In one aspect, the method includes treating polycystic kidney disease in a subject in need thereof, comprising administering an effective amount of an Hsp90 inhibitory compound shown in Table 1, or according to formula (VI) as set forth below:

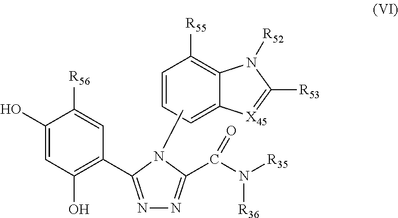

wherein:

X₄₅ is CR₅₄ or N;

R₅₆ is selected from the group consisting of —H, methyl, ethyl, isopropyl, and cyclopropyl;

R₅₂ is selected from the group consisting of —H, methyl, ethyl, n-propyl, isopropyl, n-butyl, n-pentyl, n-hexyl, —(CH₂)₂OCH₃, —CH₂C(O)OH, and —C(O)N(CH₃)₂;

R₅₃ and R₅₄ are each, independently, —H, methyl, ethyl, or isopropyl; or

R₅₃ and R₅₄ taken together with the carbon atoms to which they are attached form a phenyl, cyclohexenyl, or cyclooctenyl ring; and

R₅₅ is selected from the group consisting of —H, —OH, —OCH₃, and OCH₂CH₃;

or a pharmaceutically acceptable salt thereof.

In one aspect, the method includes treating polycystic kidney disease in a subject in need thereof, comprising administering an effective amount of an Hsp90 inhibitory compound shown in Table 1, or according to formula (VII) as set forth below:

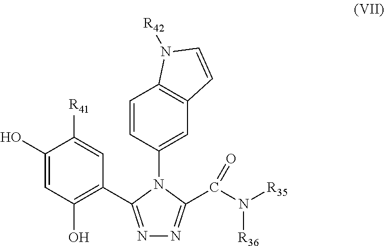

or a pharmaceutically acceptable salt thereof.

Hsp90 inhibitory compounds that may be used in the methods described herein are depicted in Table 1.

TABLE 1

| No. | Structure | Name |
|---|---|---|
| 1. | 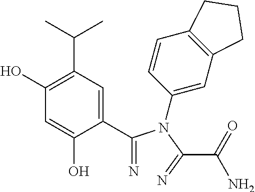 [SMILES: CC(C)c1cc(C(=N)N(C(=N)C(N)=O)c2ccc3c(c2)CCC3)c(O)cc1O] | 4-(2,3-dihydro-1H-inden-5- yl)-5-(2,4-dihydroxy-5- isopropylphenyl)-4H-1,2,4- triazole-3-carboxamide |
| 2. | 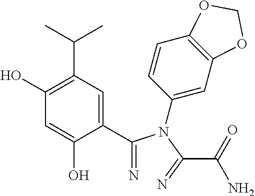 [SMILES: CC(C)c1cc(C(=N)N(C(=N)C(N)=O)c2ccc3c(c2)OCO3)c(O)cc1O] | 4-(benzo[d][1,3]dioxol-5-yl)- 5-(2,4-dihydroxy-5- isopropylphenyl)-4H-1,2,4- triazole-3-carboxamide |
| 3. | 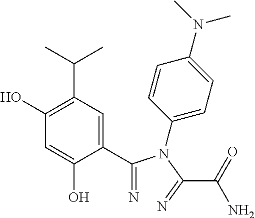 [SMILES: CC(C)c1cc(C(=N)N(C(=N)C(N)=O)c2ccc(N(C)C)cc2)c(O)cc1O] | 5-(2,4-dihydroxy-5- isopropylphenyl)-4-(4- (dimethylamino)phenyl)-4H- 1,2,4-triazole-3-carboxamide |
| 4. | 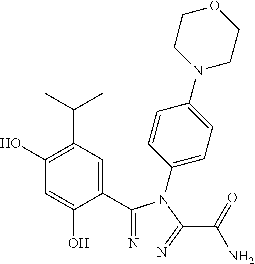 [SMILES: CC(C)c1cc(C(=N)N(C(=N)C(N)=O)c2ccc(N3CCOCC3)cc2)c(O)cc1O] | 5-(2,4-dihydroxy-5- isopropylphenyl)-4-(4- morpholinophenyl)-4H- 1,2,4-triazole-3-carboxamide |
| 5. | 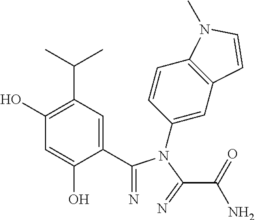 [SMILES: CC(C)c1cc(C(=N)N(C(=N)C(N)=O)c2ccc3c(ccn3C)c2)c(O)cc1O] | 5-(2,4-dihydroxy-5- isopropylphenyl)-4-(1- methyl-1H-indol-5-yl)-4H- 1,2,4-triazole-3-carboxamide |
| 6. | 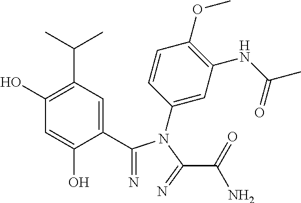 [SMILES: COc1ccc(N(C(=N)C(N)=O)C(=N)c2cc(C(C)C)c(O)cc2O)cc1NC(C)=O] | 4-(3-acetamido-4- methoxyphenyl)-5-(2,4- dihydroxy-5- isopropylphenyl)-4H-1,2,4- triazole-3-carboxamide |
| 7. | 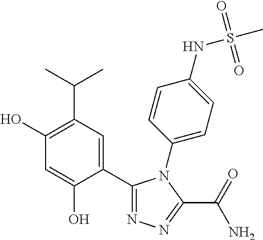 [SMILES: CC(C)c1cc(-c2nnc(C(N)=O)n2-c2ccc(NS(C)(=O)=O)cc2)c(O)cc1O] | 5-(2,4-dihydroxy-5- isopropylphenyl)-4-(4- (methylsulfonamido)phenyl)- 4H-1,2,4-triazole-3- carboxamide |
| 8. | 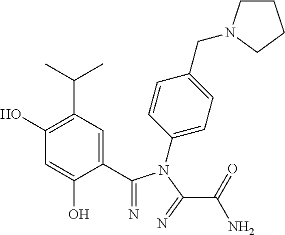 [SMILES: CC(C)c1cc(C(=N)N(C(=N)C(N)=O)c2ccc(CN3CCCC3)cc2)c(O)cc1O] | 5-(2,4-dihydroxy-5- isopropylphenyl)-4-(4- (pyrrolidin-1- ylmethyl)phenyl)-4H-1,2,4- triazole-3-carboxamide |
| 9. | 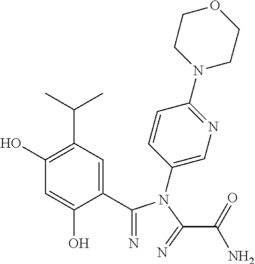 [SMILES: CC(C)c1cc(C(=N)N(C(=N)C(N)=O)c2ccc(N3CCOCC3)nc2)c(O)cc1O] | 5-(2,4-dihydroxy-5- isopropylphenyl)-4-(6- morpholinopyridin-3-yl)-4H- 1,2,4-triazole-3-carboxamide |
| 10. | 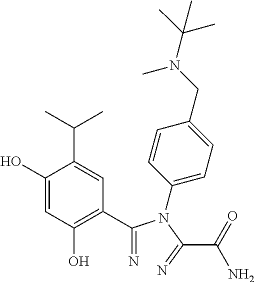 [SMILES: CC(C)c1cc(C(=N)N(C(=N)C(N)=O)c2ccc(CN(C)C(C)(C)C)cc2)c(O)cc1O] | 4-(4-((tert- butyl(methyl)amino)methyl) phenyl)-5-(2,4-dihydroxy-5- isopropylphenyl)-4H-1,2,4- triazole-3-carboxamide |
| 11. | 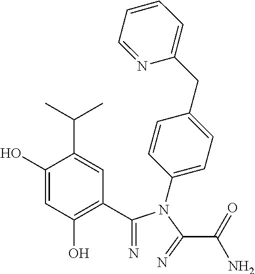 [SMILES: CC(C)c1cc(C(=N)N(C(=N)C(N)=O)c2ccc(Cc3ccccn3)cc2)c(O)cc1O] | 5-(2,4-dihydroxy-5- isopropylphenyl)-4-(4- (pyridin-2-ylmethyl)phenyl)- 4H-1,2,4-triazole-3- carboxamide |
| 12. | 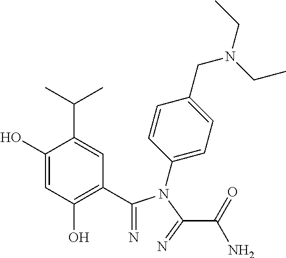 [SMILES: CCN(CC)Cc1ccc(N(C(=N)C(N)=O)C(=N)c2cc(C(C)C)c(O)cc2O)cc1] | 4-(4- ((diethylamino)methyl) phenyl)-5-(2,4-dihydroxy-5- isopropylphenyl)-4H-1,2,4- triazole-3-carboxamide |
| 13. | 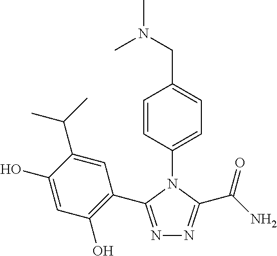 [SMILES: CC(C)c1cc(-c2nnc(C(N)=O)n2-c2ccc(CN(C)C)cc2)c(O)cc1O] | 5-(2,4-dihydroxy-5- isopropylphenyl)-4-(4- ((dimethylamino)methyl) phenyl)-4H-1,2,4-triazole-3- carboxamide |
| 14. | 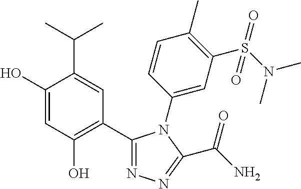 [SMILES: Cc1ccc(-n2c(C(N)=O)nnc2-c2cc(C(C)C)c(O)cc2O)cc1S(=O)(=O)N(C)C] | 5-(2,4-dihydroxy-5- isopropylphenyl)-4-(3-(N,N- dimethylsulfamoyl)-4- methylphenyl)-4H-1,2,4- triazole-3-carboxamide |
| 15. | 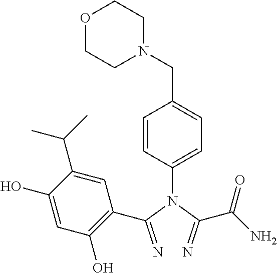 [SMILES: CC(C)c1cc(C(=N)N(C(=N)C(N)=O)c2ccc(CN3CCOCC3)cc2)c(O)cc1O] | 5-(2,4-dihydroxy-5- isopropylphenyl)-4-(4- (morpholinomethyl)phenyl)- 4H-1,2,4-triazole-3- carboxamide |
| 16. | 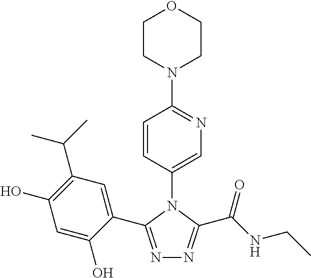 [SMILES: CCNC(=O)c1nnc(-c2cc(C(C)C)c(O)cc2O)n1-c1ccc(N2CCOCC2)nc1] | 5-(2,4-dihydroxy-5- isopropylphenyl)-N-ethyl-4- (6-morpholinopyridin-3-yl)- 4H-1,2,4-triazole-3- carboxamide |
| 17. | 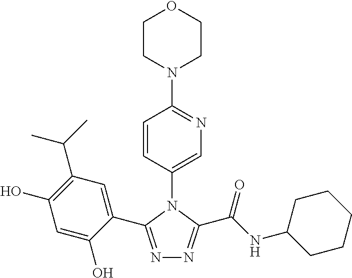 [SMILES: CC(C)c1cc(-c2nnc(C(=O)NC3CCCCC3)n2-c2ccc(N3CCOCC3)nc2)c(O)cc1O] | N-cyclohexyl-5-(2,4- dihydroxy-5- isopropylphenyl)-4-(6- morpholinopyridin-3-yl)-4H- 1,2,4-triazole-3-carboxamide |
| 18. | 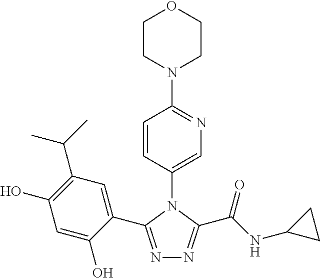 [SMILES: CC(C)c1cc(-c2nnc(C(=O)NC3CC3)n2-c2ccc(N3CCOCC3)nc2)c(O)cc1O] | N-cyclopropyl-5-(2,4- dihydroxy-5- isopropylphenyl)-4-(6- morpholinopyridin-3-yl)-4H- 1,2,4-triazole-3-carboxamide |
| 19. | 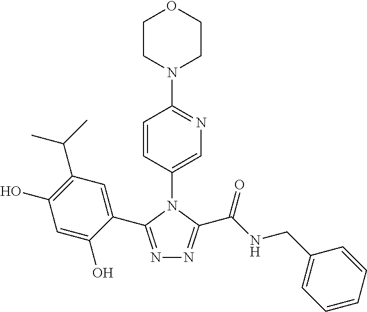 [SMILES: CC(C)c1cc(-c2nnc(C(=O)NCc3ccccc3)n2-c2ccc(N3CCOCC3)nc2)c(O)cc1O] | N-benzyl-5-(2,4-dihydroxy- 5-isopropylphenyl)-4-(6- morpholinopyridin-3-yl)-4H- 1,2,4-triazole-3-carboxamide |
| 20. | 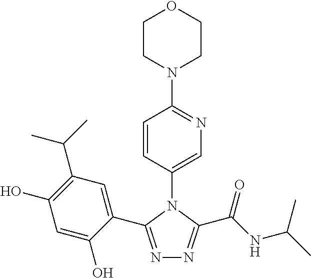 [SMILES: CC(C)NC(=O)c1nnc(-c2cc(C(C)C)c(O)cc2O)n1-c1ccc(N2CCOCC2)nc1] | 5-(2,4-dihydroxy-5- isopropylphenyl)-N- isopropyl-4-(6- morpholinopyridin-3-yl)-4H- 1,2,4-triazole-3-carboxarnide |
| 21. | 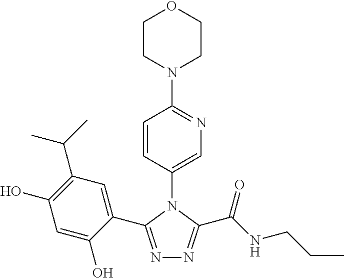 [SMILES: CCCNC(=O)c1nnc(-c2cc(C(C)C)c(O)cc2O)n1-c1ccc(N2CCOCC2)nc1] | 5-(2,4-dihydroxy-5- isopropylphenyl)-4-(6- morpholinopyridin-3-yl)-N- propyl-4H-1,2,4-triazole-3- carboxamide |
| 22. | 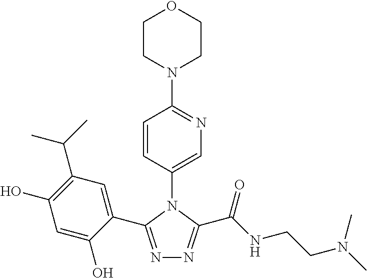 [SMILES: CC(C)c1cc(-c2nnc(C(=O)NCCN(C)C)n2-c2ccc(N3CCOCC3)nc2)c(O)cc1O] | 5-(2,4-dihydroxy-5- isopropylphenyl)-N-(2- (dimethylamino)ethyl)-4-(6- morpholinopyridin-3-yl)-4H- 1,2,4-triazole-3-carboxamide |
| 23. | 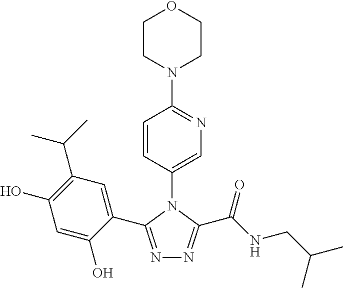 [SMILES: CC(C)CNC(=O)c1nnc(-c2cc(C(C)C)c(O)cc2O)n1-c1ccc(N2CCOCC2)nc1] | 5-(2,4-dihydroxy-5- isopropylphenyl)-N- isobutyl-4-(6- morpholinopyridin-3-yl)-4H- 1,2,4-triazole-3-carboxamide |
| 24. | 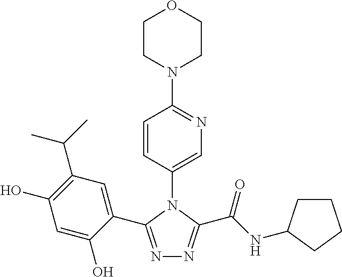 [SMILES: CC(C)c1cc(-c2nnc(C(=O)NC3CCCC3)n2-c2ccc(N3CCOCC3)nc2)c(O)cc1O] | N-cyclopentyl-5-(2,4- dihydroxy-5- isopropylphenyl)-4-(6- morpholinopyridin-3-yl)-4H- 1,2,4-triazole-3-carboxamide |
| 25. | 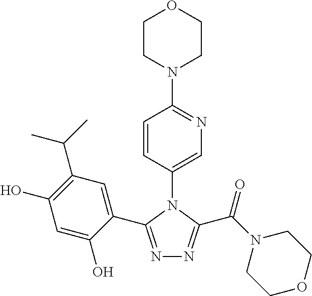 [SMILES: CC(C)c1cc(-c2nnc(C(=O)N3CCOCC3)n2-c2ccc(N3CCOCC3)nc2)c(O)cc1O] | (5-(2,4-dihydroxy-5- isopropylphenyl)-4-(6- morpholinopyridin-3-yl)-4H- 1,2,4-triazol-3- yl)(morpholino)methanone |
| 26. | 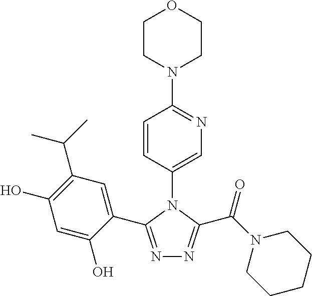 [SMILES: CC(C)c1cc(-c2nnc(C(=O)N3CCCCC3)n2-c2ccc(N3CCOCC3)nc2)c(O)cc1O] | (5-(2,4-dihydroxy-5- isopropylphenyl)-4-(6- morpholinopyridin-3-yl)-4H- 1,2,4-triazol-3-yl)(piperidin- 1-yl)methanone |
| 27. | 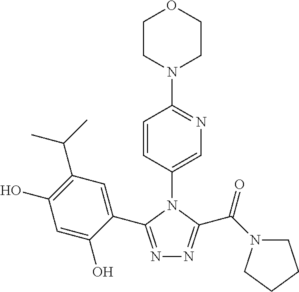 [SMILES: CC(C)c1cc(-c2nnc(C(=O)N3CCCC3)n2-c2ccc(N3CCOCC3)nc2)c(O)cc1O] | (5-(2,4-dihydroxy-5- isopropylphenyl)-4-(6- morpholinopyridin-3-yl)-4H- 1,2,4-triazol-3-yl)(pyrrolidin- 1-yl)methanone |
| 28. | 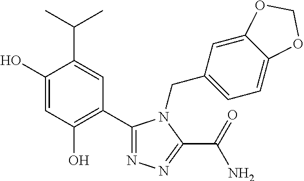 [SMILES: CC(C)c1cc(-c2nnc(C(N)=O)n2Cc2ccc3c(c2)OCO3)c(O)cc1O] | 4-(benzo[d][1,3]dioxol-5- ylmethyl)-5-(2,4-dihydroxy- 5-isopropylphenyl)-4H-1,2,4- triazole-3-carboxamide |
| 29. | 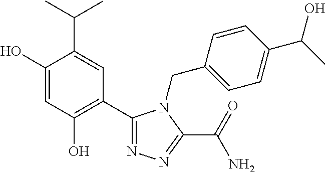 [SMILES: CC(C)c1cc(-c2nnc(C(N)=O)n2Cc2ccc(C(C)O)cc2)c(O)cc1O] | 5-(2,4-dihydroxy-5- isopropylphenyl)-4-(4-(1- hydroxyethyl)benzyl)-4H- 1,2,4-triazole-3-carboxamide |
| 30. | 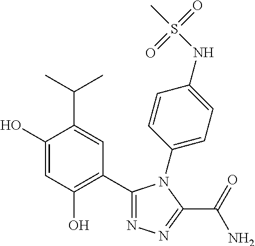 [SMILES: CC(C)c1cc(-c2nnc(C(N)=O)n2-c2ccc(NS(C)(=O)=O)cc2)c(O)cc1O] | 5-(2,4-dihydroxy-5- isopropylphenyl)-4-(4- (methylsulfonamido)phenyl)- 4H-1,2,4-triazole-3- carboxamide |
| 31. | 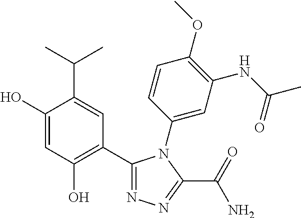 [SMILES: COc1ccc(-n2c(C(N)=O)nnc2-c2cc(C(C)C)c(O)cc2O)cc1NC(C)=O] | 4-(3-acetamido-4- methoxyphenyl)-5-(2,4- dihydroxy-5- isopropylphenyl)-4H-1,2,4- triazole-3-carboxamide |
| 32. | 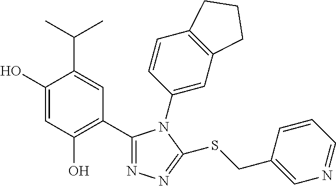 [SMILES: CC(C)c1cc(-c2nnc(SCc3cccnc3)n2-c2ccc3c(c2)CCC3)c(O)cc1O] | 4-(4-(2,3-dihydro-1H-inden- 5-yl)-5-(pyridin-3- ylmethylthio)-4H-1,2,4- triazol-3-yl)-6- isopropylbenzene-1,3-diol |
| 33. | 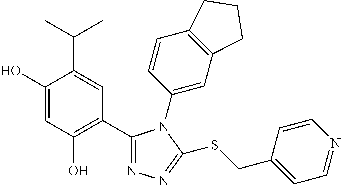 [SMILES: CC(C)c1cc(-c2nnc(SCc3ccncc3)n2-c2ccc3c(c2)CCC3)c(O)cc1O] | 4-(4-(2,3-dihydro-1H-inden- 5-yl)-5-(pyridin-4- ylmethylthio)-4H-1,2,4- triazol-3-yl)-6- isopropylbenzene-1,3-diol |
| 34. | 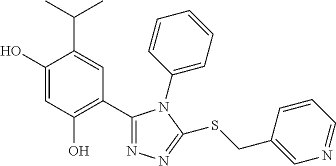 [SMILES: CC(C)c1cc(-c2nnc(SCc3cccnc3)n2-c2ccccc2)c(O)cc1O] | 4-isopropyl-6-(4-phenyl-5- (pyridin-3-ylmethylthio)-4H- 1,2,4-triazol-3-yl)benzene- 1,3-diol |
| 35. | 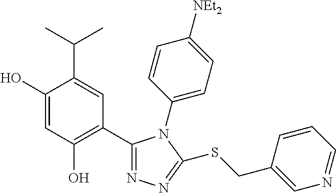 | 4-(4-(4- (diethylamino)phenyl)-5- (pyridin-3-ylmethylthio)-4H- 1,2,4-triazol-3-yl)-6- isopropylbenzene-1,3-diol |
| 36. | 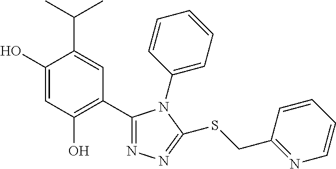 [SMILES: CC(C)c1cc(-c2nnc(SCc3ccccn3)n2-c2ccccc2)c(O)cc1O] | 4-isopropyl-6-(4-phenyl-5- (pyridin-2-ylmethylthio)-4H- 1,2,4-triazol-3-yl)benzene- 1,3-diol |
| 37. | 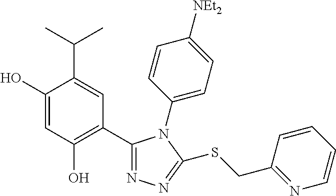 | 4-(4-(4- (diethylamino)phenyl)-5- (pyridin-2-ylmethylthio)-4H- 1,2,4-triazol-3-yl)-6- isopropylbenzene-1,3-diol |
| 38. | 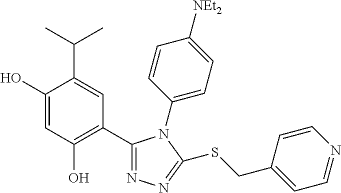 | 4-(4-(4- (diethylamino)phenyl)-5- (pyridin-4-ylmethylthio)-4H- 1,2,4-triazol-3-yl)-6- isopropylbenzene-1,3-diol |
| 39. | 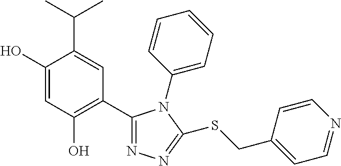 [SMILES: CC(C)c1cc(-c2nnc(SCc3ccncc3)n2-c2ccccc2)c(O)cc1O] | 4-isopropyl-6-(4-phenyl-5- (pyridin-4-ylmethylthio)-4H- 1,2,4-triazol-3-yl)benzene- 1,3-diol |
| 40. | 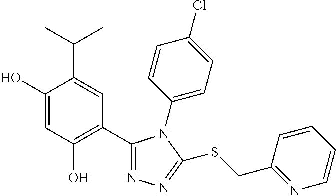 [SMILES: CC(C)c1cc(-c2nnc(SCc3ccccn3)n2-c2ccc(Cl)cc2)c(O)cc1O] | 4-(4-(4-chlorophenyl)-5- (pyridin-2-ylmethylthio)-4H- 1,2,4-triazol-3-yl)-6- isopropylbenzene-1,3-diol |
| 41. | 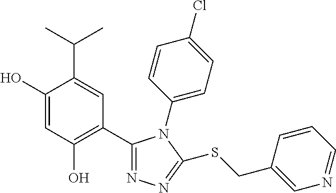 [SMILES: CC(C)c1cc(-c2nnc(SCc3cccnc3)n2-c2ccc(Cl)cc2)c(O)cc1O] | 4-(4-(4-chlorophenyl)-5- (pyridin-3-ylmethylthio)-4H- 1,2,4-triazol-3-yl)-6- isopropylbenzene-1,3-diol |
| 42. | 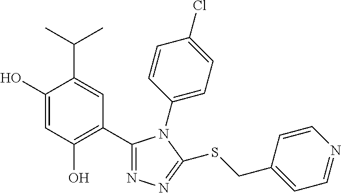 [SMILES: CC(C)c1cc(-c2nnc(SCc3ccncc3)n2-c2ccc(Cl)cc2)c(O)cc1O] | 4-(4-(4-chlorophenyl)-5- (pyridin-4-ylmethylthio)-4H- 1,2,4-triazol-3-yl)-6- isopropylbenzene-1,3-diol |
| 43. | 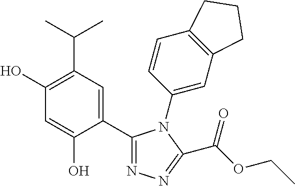 [SMILES: CCOC(=O)c1nnc(-c2cc(C(C)C)c(O)cc2O)n1-c1ccc2c(c1)CCC2] | ethyl 4-(2,3-dihydro-1H- inden-5-yl)-5-(2,4- dihydroxy-5- isopropylphenyl)-4H-1,2,4- triazole-3-carboxylate |
| 44. | 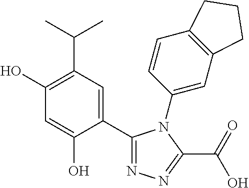 [SMILES: CC(C)c1cc(-c2nnc(C(=O)O)n2-c2ccc3c(c2)CCC3)c(O)cc1O] | 4-(2,3-dihydro-1H-inden-5- yl)-5-(2,4-dihydroxy-5- isopropylphenyl)-4H-1,2,4- triazole-3-carboxylic acid |
| 45. | 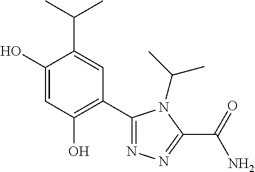 [SMILES: CC(C)c1cc(-c2nnc(C(N)=O)n2C(C)C)c(O)cc1O] | 5-(2,4-dihydroxy-5- isopropylphenyl)-4- isopropyl-4H-1,2,4-triazole- 3-carboxamide |
| 46. | 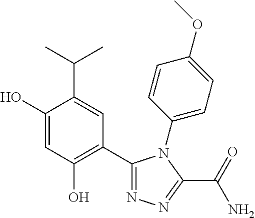 [SMILES: COc1ccc(-n2c(C(N)=O)nnc2-c2cc(C(C)C)c(O)cc2O)cc1] | 5-(2,4-dihydroxy-5- isopropylphenyl)-4-(4- methoxyphenyl)-4H-1,2,4- triazole-3-carboxamide |
| 47. | 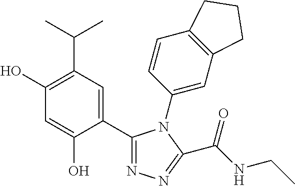 [SMILES: CCNC(=O)c1nnc(-c2cc(C(C)C)c(O)cc2O)n1-c1ccc2c(c1)CCC2] | 4-(2,3-dihydro-1H-inden-5- yl)-5-(2,4-dihydroxy-5- isopropylphenyl)-N-ethyl- 4H-1,2,4-triazole-3- carboxamide |
| 48. | 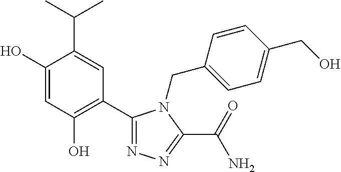 [SMILES: CC(C)c1cc(-c2nnc(C(N)=O)n2Cc2ccc(CO)cc2)c(O)cc1O] | 5-(2,4-dihydroxy-5- isopropylphenyl)-4-(4- (hydroxymethyl)benzyl)-4H- 1,2,4-triazole-3-carboxamide |
| 49. | 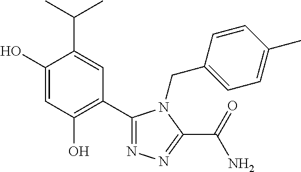 [SMILES: Cc1ccc(Cn2c(C(N)=O)nnc2-c2cc(C(C)C)c(O)cc2O)cc1] | 5-(2,4-dihydroxy-5- isopropylphenyl)-4-(4- methylbenzyl)-4H-1,2,4- triazole-3-carboxamide |
| 50. | 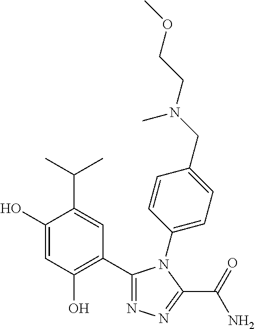 [SMILES: COCCN(C)Cc1ccc(-n2c(C(N)=O)nnc2-c2cc(C(C)C)c(O)cc2O)cc1] | 5-(2,4-dihydroxy-5- isopropylphenyl)-4-(4-(((2- methoxyethyl)(methyl) amino)methyl)phenyl)-4H- 1,2,4-triazole-3-carboxamide |
| 51. | 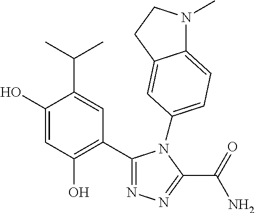 [SMILES: CC(C)c1cc(-c2nnc(C(N)=O)n2-c2ccc3c(c2)CCN3C)c(O)cc1O] | 5-(2,4-dihydroxy-5- isopropylphenyl)-4-(1- methylindolin-5-yl)-4H- 1,2,4-triazole-3-carboxamide |
| 52. | 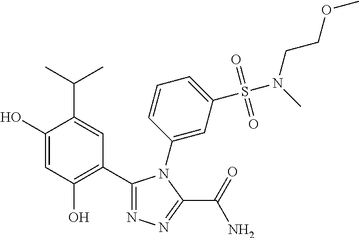 [SMILES: COCCN(C)S(=O)(=O)c1cccc(-n2c(C(N)=O)nnc2-c2cc(C(C)C)c(O)cc2O)c1] | 5-(2,4-dihydroxy-5- isopropylphenyl)-4-(3-(N-(2- methoxyethyl)-N- methylsulfamoyl)phenyl)- 4H-1,2,4-triazole-3- carboxamide |
| 53. | 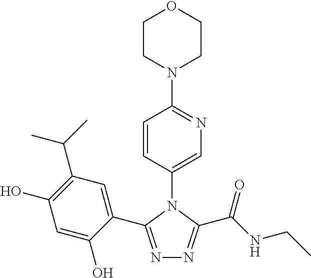 [SMILES: CCNC(=O)c1nnc(-c2cc(C(C)C)c(O)cc2O)n1-c1ccc(N2CCOCC2)nc1] | 5-(2,4-dihydroxy-5- isopropylphenyl)-N-ethy]-4- (6-morpholinopyridin-3-yl)- 4H-1,2,4-triazole-3- carboxamide |
| 54. | 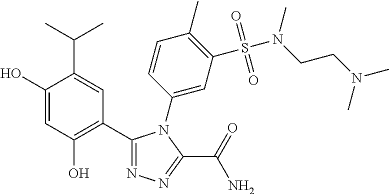 [SMILES: Cc1ccc(-n2c(C(N)=O)nnc2-c2cc(C(C)C)c(O)cc2O)cc1S(=O)(=O)N(C)CCN(C)C] | 5-(2,4-dihydroxy-5- isopropylphenyl)-4-(3-(N-(2- (dimethylamino)ethyl)-N- methylsulfamoyl)-4- methylphenyl)-4H-1,2,4- triazole-3-carboxamide |
| 55. | 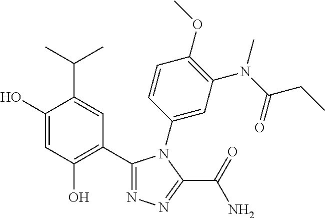 [SMILES: CCC(=O)N(C)c1cc(-n2c(C(N)=O)nnc2-c2cc(C(C)C)c(O)cc2O)ccc1OC] | 5-(2,4-dihydroxy-5- isopropylphenyl)-4-(4- methoxy-3-(N- methylpropionamido) phenyl)-4H-1,2,4-triazole- 3-carboxamide |
| 56. | 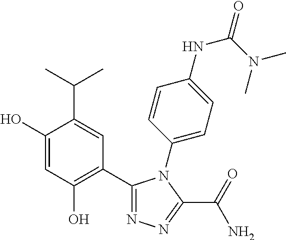 [SMILES: CC(C)c1cc(-c2nnc(C(N)=O)n2-c2ccc(NC(=O)N(C)C)cc2)c(O)cc1O] | 5-(2,4-dihydroxy-5- isopropylphenyl)-4-(4-(3,3- dimethylureido)phenyl)-4H- 1,2,4-triazole-3-carboxamide |
| 57. | 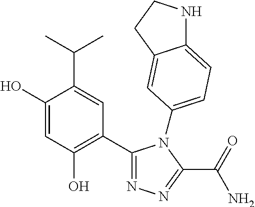 [SMILES: CC(C)c1cc(-c2nnc(C(N)=O)n2-c2ccc3c(c2)CCN3)c(O)cc1O] | 5-(2,4-dihydroxy-5- isopropylphenyl)-4-(indolin- 5-yl)-4H-1,2,4-triazole-3- carboxamide |
| 58. | 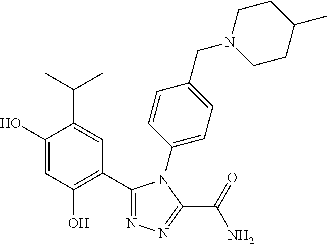 [SMILES: CC1CCN(Cc2ccc(-n3c(C(N)=O)nnc3-c3cc(C(C)C)c(O)cc3O)cc2)CC1] | 5-(2,4-dihydroxy-5- isopropylphenyl)-4-(4-((4- methylpiperidin-1- yl)methyl)phenyl)-4H-1,2,4- triazole-3-carboxamide |
| 59. | 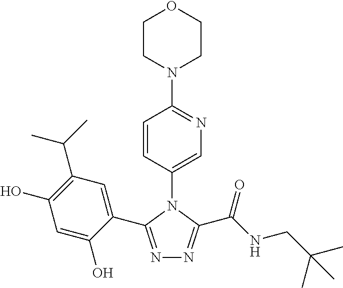 [SMILES: CC(C)c1cc(-c2nnc(C(=O)NCC(C)(C)C)n2-c2ccc(N3CCOCC3)nc2)c(O)cc1O] | 5-(2,4-dihydroxy-5- isopropylphenyl)-4-(6- morpholinopyridin-3-yl)-N- neopentyl-4H-1,2,4-triazole- 3-carboxamide |
| 60. | 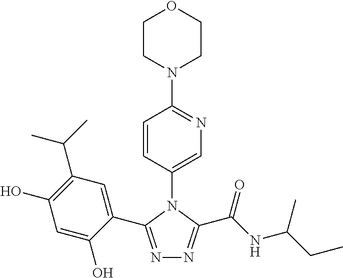 [SMILES: CCC(C)NC(=O)c1nnc(-c2cc(C(C)C)c(O)cc2O)n1-c1ccc(N2CCOCC2)nc1] | N-sec-butyl-5-(2,4- dihydroxy-5- isopropylphenyl)-4-(6- morpholinopyridin-3-yl)-4H- 1,2,4-triazole-3-carboxamide |
| 61. | 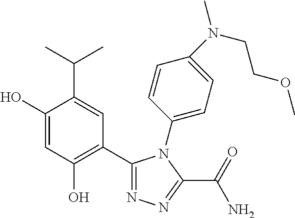 [SMILES: COCCN(C)c1ccc(-n2c(C(N)=O)nnc2-c2cc(C(C)C)c(O)cc2O)cc1] | 5-(2,4-dihydroxy-5- isopropylphenyl)-4-(4-((2- methoxyethyl)(methyl) amino)phenyl)-4H-1,2,4- triazole-3-carboxamide |
| 62. | 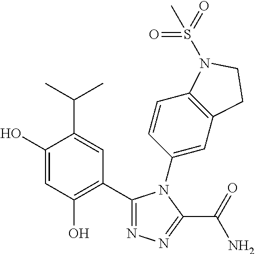 [SMILES: CC(C)c1cc(-c2nnc(C(N)=O)n2-c2ccc3c(c2)CCN3S(C)(=O)=O)c(O)cc1O] | 5-(2,4-dihydroxy-5- isopropylphenyl)-4-(1- (methylsulfonyl)indolin-5- yl)-4H-1,2,4-triazole-3- carboxamide |
| 63. | 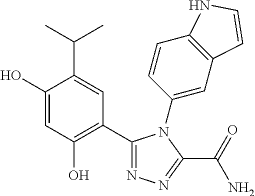 [SMILES: CC(C)c1cc(-c2nnc(C(N)=O)n2-c2ccc3[nH]ccc3c2)c(O)cc1O] | 5-(2,4-dihydroxy-5- isopropylphenyl)-4-(1H- indol-5-yl)-4H-1,2,4-triazole- 3-carboxamide |
| 64. | 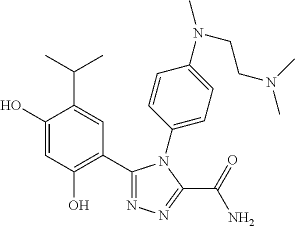 [SMILES: CC(C)c1cc(-c2nnc(C(N)=O)n2-c2ccc(N(C)CCN(C)C)cc2)c(O)cc1O] | 5-(2,4-dihydroxy-5- isopropylphenyl)-4-(4-((2- (dimethylamino)ethyl) (methyl)amino)phenyl)-4H- 1,2,4-triazole-3-carboxamide |
| 65. | 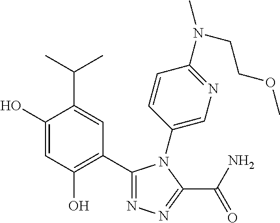 [SMILES: COCCN(C)c1ccc(-n2c(C(N)=O)nnc2-c2cc(C(C)C)c(O)cc2O)cn1] | 5-(2,4-dihydroxy-5- isopropylphenyl)-4-(6-((2- methoxyethyl)(methyl) amino)pyridin-3-yl)-4H- 1,2,4-triazole-3-carboxamide |
| 66. | 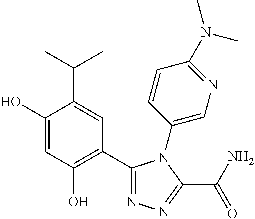 [SMILES: CC(C)c1cc(-c2nnc(C(N)=O)n2-c2ccc(N(C)C)nc2)c(O)cc1O] | 5-(2,4-dihydroxy-5- isopropylphenyl)-4-(6- (dimethylamino)pyridin-3- yl)-4H-1,2,4-triazole-3- carboxamide |
| 67. | 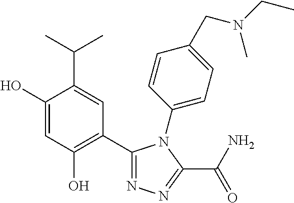 [SMILES: CCN(C)Cc1ccc(-n2c(C(N)=O)nnc2-c2cc(C(C)C)c(O)cc2O)cc1] | 5-(2,4-dihydroxy-5- isopropylphenyl)-4-(4- ((ethyl(methyl)amino)methyl) phenyl)-4H-1,2,4-triazole-3- carboxamide |
| 68. | 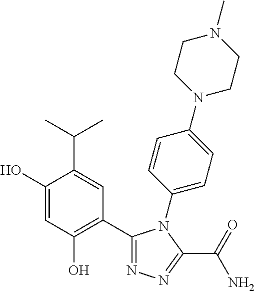 [SMILES: CC(C)c1cc(-c2nnc(C(N)=O)n2-c2ccc(N3CCN(C)CC3)cc2)c(O)cc1O] | 5-(2,4-dihydroxy-5- isopropylphenyl)-4-(4-(4- methylpiperazin-1- yl)phenyl)-4H-1,2,4-triazole- 3-carboxamide |
| 69. | 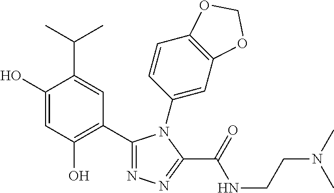 [SMILES: CC(C)c1cc(-c2nnc(C(=O)NCCN(C)C)n2-c2ccc3c(c2)OCO3)c(O)cc1O] | 4-(benzo[d][1,3]dioxol-5-yl)- 5-(2,4-dihydroxy-5- isopropylphenyl)-N-(2- (dimethy!amino)ethyl)-4H- 1,2,4-triazole-3-carboxamide |
| 70. | 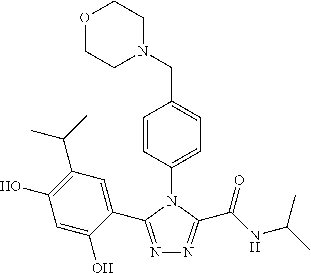 [SMILES: CC(C)NC(=O)c1nnc(-c2cc(C(C)C)c(O)cc2O)n1-c1ccc(CN2CCOCC2)cc1] | 5-(2,4-dihydroxy-5- isopropylphenyl)-N- isopropyl-4-(4- (morpholinomethyl)phenyl)- 4H-1,2,4-triazole-3- carboxamide |
| 71. | 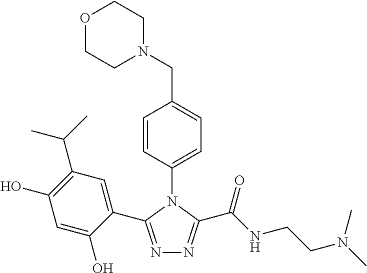 [SMILES: CC(C)c1cc(-c2nnc(C(=O)NCCN(C)C)n2-c2ccc(CN3CCOCC3)cc2)c(O)cc1O] | 5-(2,4-dihydroxy-5- isopropylphenyl)-N-(2- (dimethylamino)ethyl)-4-(4- (morpholinomethyl)phenyl)- 4H-1,2,4-triazole-3- carboxamide |
| 72. | 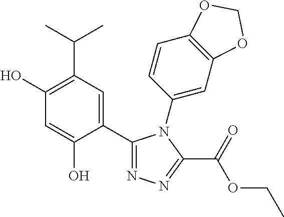 [SMILES: CCOC(=O)c1nnc(-c2cc(C(C)C)c(O)cc2O)n1-c1ccc2c(c1)OCO2] | ethyl 4-(benzo[d][1,3]dioxol- 5-yl)-5-(2,4-dihydroxy-5- isopropylphenyl)-4H-1,2,4- triazole-3-carboxylate |
| 73. | 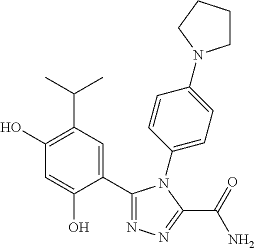 [SMILES: CC(C)c1cc(-c2nnc(C(N)=O)n2-c2ccc(N3CCCC3)cc2)c(O)cc1O] | 5-(2,4-dihydroxy-5- isopropylphenyl)-4-(4- (pyrrolidin-1-yl)phenyl)-4H- 1,2,4-triazole-3-carboxamide |
| 74. | 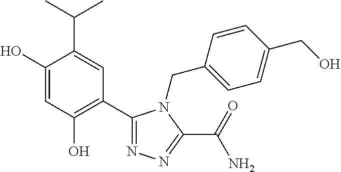 [SMILES: CC(C)c1cc(-c2nnc(C(N)=O)n2Cc2ccc(CO)cc2)c(O)cc1O] | 5-(2,4-dihydroxy-5- isopropylphenyl)-4-(4- (hydroxymethyl)benzyl)-4H- 1,2,4-triazole-3-carboxamide |
| 75. | 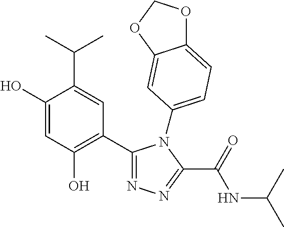 [SMILES: CC(C)NC(=O)c1nnc(-c2cc(C(C)C)c(O)cc2O)n1-c1ccc2c(c1)OCO2] | 4-(benzo[d][1,3]dioxol-5-yl)- 5-(2,4-dihydroxy-5- isopropylphenyl)-N- isopropyl-4H-1,2,4-triazole- 3-carboxamide |
| 76. | 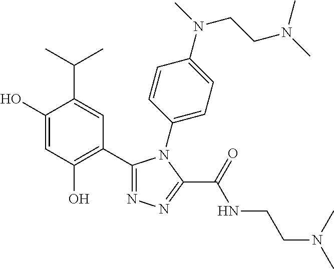 [SMILES: CC(C)c1cc(-c2nnc(C(=O)NCCN(C)C)n2-c2ccc(N(C)CCN(C)C)cc2)c(O)cc1O] | 5-(2,4-dihydroxy-5- isopropylphenyl)-N-(2- (dimethylamino)ethyl)-4-(4- ((2- (dimethylamino)ethyl) (methyl)amino)phenyl)- 4H-1,2,4-triazole-3- carboxamide |
| 77. | 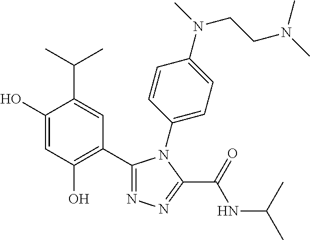 [SMILES: CC(C)NC(=O)c1nnc(-c2cc(C(C)C)c(O)cc2O)n1-c1ccc(N(C)CCN(C)C)cc1] | 5-(2,4-dihydroxy-5- isopropylphenyl)-4-(4-((2- (dimethylamino)ethyl) (methyl)amino)phenyl)-N- isopropyl-4H-1,2,4-triazole- 3-carboxamide |
| 78. | 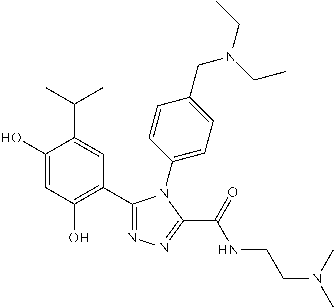 [SMILES: CCN(CC)Cc1ccc(-n2c(C(=O)NCCN(C)C)nnc2-c2cc(C(C)C)c(O)cc2O)cc1] | 4-(4- ((diethylamino)methyl) phenyl)-5-(2,4-dihydroxy-5- isopropylphenyl)-N-(2- (dimethylamino)ethyl)-4H- 1,2,4-triazole-3-carboxamide |
| 79. | 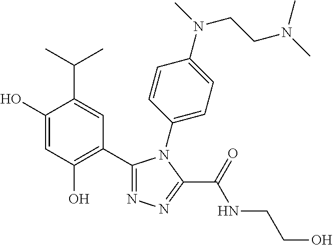 [SMILES: CC(C)c1cc(-c2nnc(C(=O)NCCO)n2-c2ccc(N(C)CCN(C)C)cc2)c(O)cc1O] | 5-(2,4-dihydroxy-5- isopropylphenyl)-4-(4-((2- (dimethylamino)ethyl) (methyl)amino)phenyl)-N- (2-hydroxyethyl)-4H-1,2,4- triazole-3-carboxamide |
| 80. | 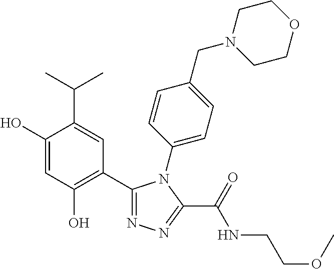 [SMILES: COCCNC(=O)c1nnc(-c2cc(C(C)C)c(O)cc2O)n1-c1ccc(CN2CCOCC2)cc1] | 5-(2,4-dihydroxy-5- isopropylphenyl)-N-(2- methoxyethyl)-4-(4- (morpholinomethyl)phenyl)- 4H-1,2,4-triazole-3- carboxamide |
| 81. | 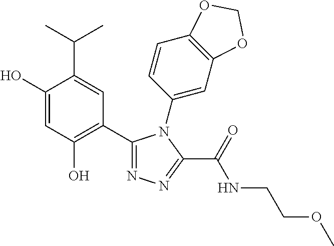 [SMILES: COCCNC(=O)c1nnc(-c2cc(C(C)C)c(O)cc2O)n1-c1ccc2c(c1)OCO2] | 4-(benzo[d][1,3]dioxol-5-yl)- 5-(2,4-dihydroxy-5- isopropylphenyl)-N-(2- methoxyethyl)-4H-1,2,4- triazole-3-carboxamide |
| 82. | 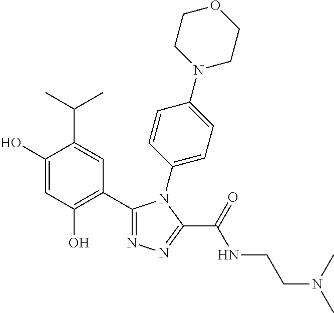 [SMILES: CC(C)c1cc(-c2nnc(C(=O)NCCN(C)C)n2-c2ccc(N3CCOCC3)cc2)c(O)cc1O] | 5-(2,4-dihydroxy-5- isopropylphenyl)-N-(2- (dimethylamino)ethyl)-4-(4- morpholinophenyl)-4H- 1,2,4-triazole-3-carboxamide |
| 83. | 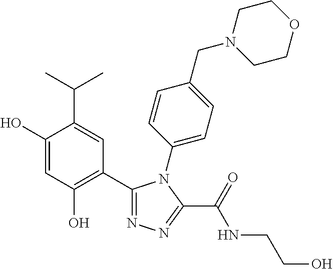 [SMILES: CC(C)c1cc(-c2nnc(C(=O)NCCO)n2-c2ccc(CN3CCOCC3)cc2)c(O)cc1O] | 5-(2,4-dihydroxy-5- isopropylphenyl)-N-(2- hydroxyethyl)-4-(4- (morpholinomethyl)phenyl)- 4H-1,2,4-triazole-3- carboxamide |
| 84. | 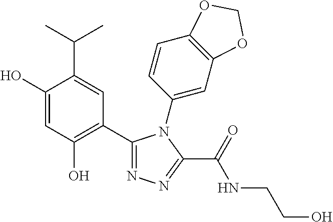 [SMILES: CC(C)c1cc(-c2nnc(C(=O)NCCO)n2-c2ccc3c(c2)OCO3)c(O)cc1O] | 4-(benzo[d][1,3]dioxol-5-yl)- 5-(2,4-dihydroxy-5- isopropylphenyl)-N-(2- hydroxyethyl)-4H-1,2,4- triazole-3-carboxamide |
| 85. | 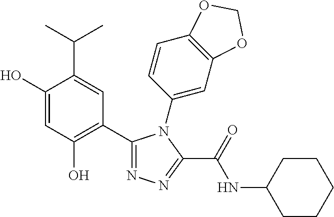 [SMILES: CC(C)c1cc(-c2nnc(C(=O)NC3CCCCC3)n2-c2ccc3c(c2)OCO3)c(O)cc1O] | 4-(benzo[d][1,3]dioxol-5-yl)- N-cyclohexyl-5-(2,4- dihydroxy-5- isopropylphenyl)-4H-1,2,4- triazole-3-carboxamide |
| 86. | 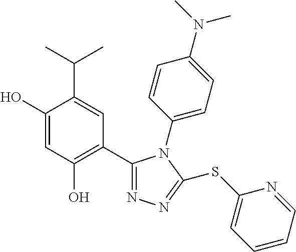 [SMILES: CC(C)c1cc(-c2nnc(Sc3ccccn3)n2-c2ccc(N(C)C)cc2)c(O)cc1O] | 4-(4-(4- (dimethylamino)phenyl)-5- (pyridin-2-ylthio)-4H-1,2,4- triazol-3-yl)-6- isopropylbenzene-1,3-diol |
| 87. | 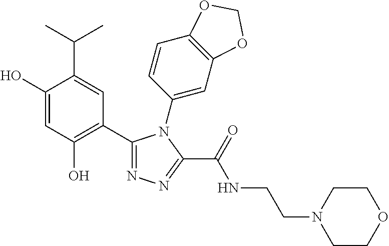 [SMILES: CC(C)c1cc(-c2nnc(C(=O)NCCN3CCOCC3)n2-c2ccc3c(c2)OCO3)c(O)cc1O] | 4-(benzo[d][1,3]dioxol-5-yl)- 5-(2,4-dihydroxy-5- isopropylphenyl)-N-(2- morpholinoethyl)-4H-1,2,4- triazole-3-carboxamide |
| 88. | 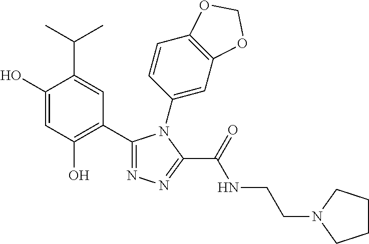 [SMILES: CC(C)c1cc(-c2nnc(C(=O)NCCN3CCCC3)n2-c2ccc3c(c2)OCO3)c(O)cc1O] | 4-(benzo[d][1,3]dioxol-5-yl)- 5-(2,4-dihydroxy-5- isopropylphenyl)-N-(2- (pyrrolidin-1-yl)ethyl)-4H- 1,2,4-triazole-3-carboxamide |
| 89. | 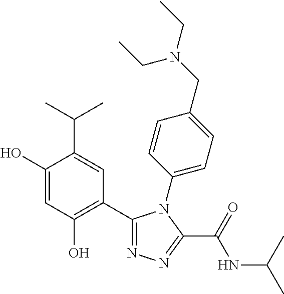 [SMILES: CCN(CC)Cc1ccc(-n2c(C(=O)NC(C)C)nnc2-c2cc(C(C)C)c(O)cc2O)cc1] | 4-(4- ((diethylamino)methyl) phenyl)-5-(2,4-dihydroxy-5- isopropylphenyl)-N- isopropyl-4H-1,2,4-triazole- 3-carboxamide |
| 90. | 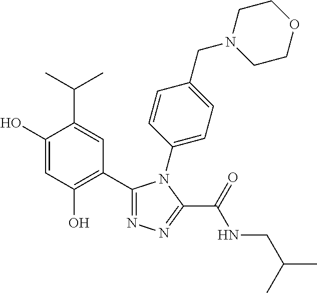 [SMILES: CC(C)CNC(=O)c1nnc(-c2cc(C(C)C)c(O)cc2O)n1-c1ccc(CN2CCOCC2)cc1] | 5-(2,4-dihydroxy-5- isopropylphenyl)-N- isobutyl-4-(4- (morpholinomethyl)phenyl)- 4H-1,2,4-triazole-3- carboxamide |
| 91. | 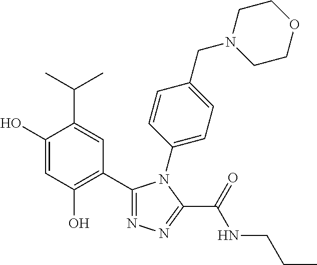 [SMILES: CCCNC(=O)c1nnc(-c2cc(C(C)C)c(O)cc2O)n1-c1ccc(CN2CCOCC2)cc1] | 5-(2,4-dihydroxy-5- isopropylphenyl)-4-(4- (morpholinomethyl)phenyl)- N-propyl-4H-1,2,4-triazole- 3-carboxamide |
| 92. | 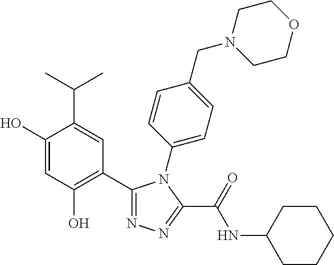 [SMILES: CC(C)c1cc(-c2nnc(C(=O)NC3CCCCC3)n2-c2ccc(CN3CCOCC3)cc2)c(O)cc1O] | N-cyclohexyl-5-(2,4- dihydroxy-5- isopropylphenyl)-4-(4- (morpholinomethyl)phenyl)- 4H-1,2,4-triazole-3- carboxamide |
| 93. | 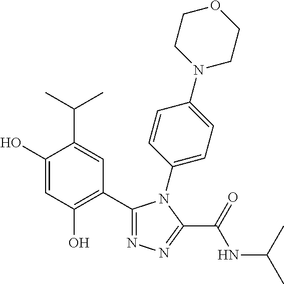 [SMILES: CC(C)NC(=O)c1nnc(-c2cc(C(C)C)c(O)cc2O)n1-c1ccc(N2CCOCC2)cc1] | 5-(2,4-dihydroxy-5- isopropylphenyl)-N- isopropyl-4-(4- morpholinophenyl)-4H- 1,2,4-triazole-3-carboxamide |
| 94. | 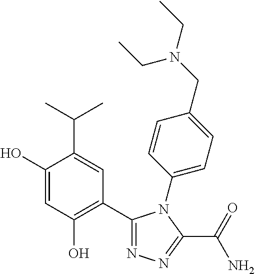 [SMILES: CCN(CC)Cc1ccc(-n2c(C(N)=O)nnc2-c2cc(C(C)C)c(O)cc2O)cc1] | 4-(4- ((diethylamino)methyl) phenyl)-5-(2,4-dihydroxy-5- isopropylphenyl)-4H-1,2,4- triazole-3-carboxamide |
| 95. | 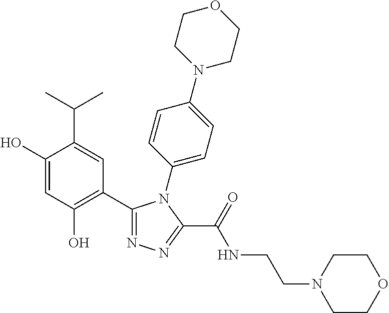 [SMILES: CC(C)c1cc(-c2nnc(C(=O)NCCN3CCOCC3)n2-c2ccc(N3CCOCC3)cc2)c(O)cc1O] | 5-(2,4-dihydroxy-5- isopropylphenyl)-N-(2- morpholinoethyl)-4-(4- morpholinophenyl)-4H- 1,2,4-triazole-3-carboxamide |
| 96. | 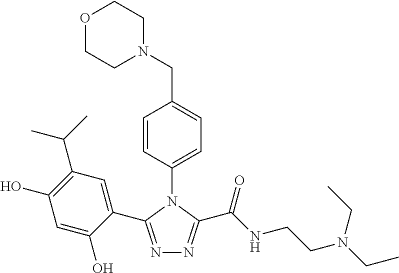 [SMILES: CCN(CC)CCNC(=O)c1nnc(-c2cc(C(C)C)c(O)cc2O)n1-c1ccc(CN2CCOCC2)cc1] | N-(2-(diethylamino)ethyl)-5- (2,4-dihydroxy-5- isopropylphenyl)-4-(4- (morpholinomethyl)phenyl)- 4H-1,2,4-triazole-3- carboxamide |
| 97. | 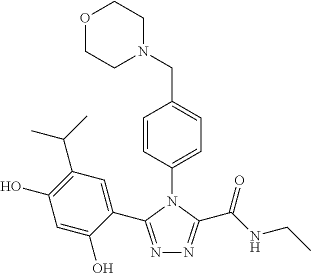 [SMILES: CCNC(=O)c1nnc(-c2cc(C(C)C)c(O)cc2O)n1-c1ccc(CN2CCOCC2)cc1] | 5-(2,4-dihydroxy-5- isopropylphenyl)-N-ethyl-4- (4- (morpholinomethyl)phenyl)- 4H-1,2,4-triazole-3- carboxamide |
| 98. | 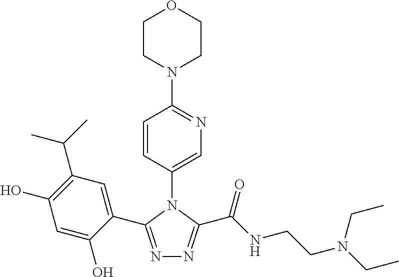 [SMILES: CCN(CC)CCNC(=O)c1nnc(-c2cc(C(C)C)c(O)cc2O)n1-c1ccc(N2CCOCC2)nc1] | N-(2-(diethylamino)ethyl)-5- (2,4-dihydroxy-5- isopropylphenyl)-4-(6- morpholinopyridin-3-yl)-4H- 1,2,4-triazole-3-carboxamide |
| 99. | 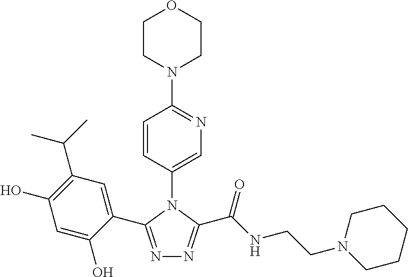 [SMILES: CC(C)c1cc(-c2nnc(C(=O)NCCN3CCCCC3)n2-c2ccc(N3CCOCC3)nc2)c(O)cc1O] | 5-(2,4-dihydroxy-5- isopropylphenyl)-4-(6- morpholinopyridin-3-yl)-N- (2-(piperidin-1-yl)ethyl)-4H- 1,2,4-triazole-3-carboxamide |
| 100. | 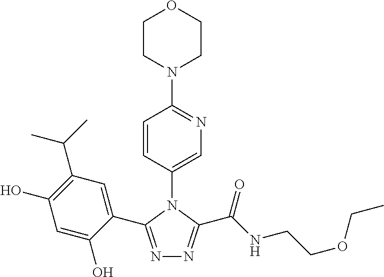 [SMILES: CCOCCNC(=O)c1nnc(-c2cc(C(C)C)c(O)cc2O)n1-c1ccc(N2CCOCC2)nc1] | 5-(2,4-dihydroxy-5- isopropylphenyl)-N-(2- ethoxyethyl)-4-(6- morpholinopyridin-3-yl)-4H- 1,2,4-triazole-3-carboxamide |
| 101. | 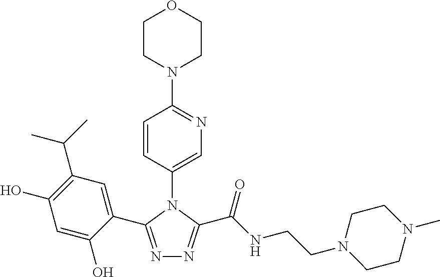 [SMILES: CC(C)c1cc(-c2nnc(C(=O)NCCN3CCN(C)CC3)n2-c2ccc(N3CCOCC3)nc2)c(O)cc1O] | 5-(2,4-dihydroxy-5- isopropylphenyl)-N-(2-(4- methylpiperazin-1-yl)ethyl)- 4-(6-morpholinopyridin-3- yl)-4H-1,2,4-triazole-3- carboxamide |
| 102. | 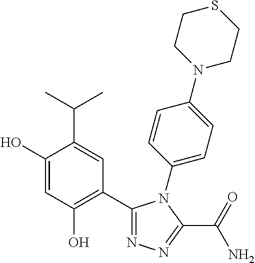 [SMILES: CC(C)c1cc(-c2nnc(C(N)=O)n2-c2ccc(N3CCSCC3)cc2)c(O)cc1O] | 5-(2,4-dihydroxy-5- isopropylphenyl)-4-(4- thiomorpholinophenyl)-4H- 1,2,4-triazole-3-carboxamide |
| 103. | 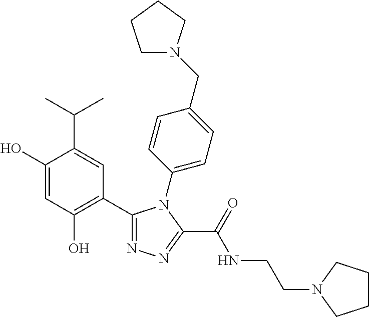 [SMILES: CC(C)c1cc(-c2nnc(C(=O)NCCN3CCCC3)n2-c2ccc(CN3CCCC3)cc2)c(O)cc1O] | 5-(2,4-dihydroxy-5- isopropylphenyl)-N-(2- (pyrrolidin-1-yl)ethyl)-4-(4- (pyrrolidin-1- ylmethyl)phenyl)-4H-1,2,4- triazole-3-carboxamide |
| 104. | 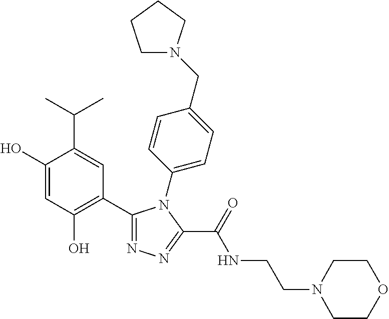 [SMILES: CC(C)c1cc(-c2nnc(C(=O)NCCN3CCOCC3)n2-c2ccc(CN3CCCC3)cc2)c(O)cc1O] | 5-(2,4-dihydroxy-5- isopropylphenyl)-N-(2- morpholinoethyl)-4-(4- (pyrrolidin-1- ylmethyl)phenyl)-4H-1,2,4- triazole-3-carboxamide |
| 105. | 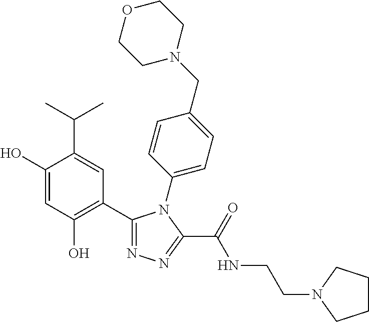 [SMILES: CC(C)c1cc(-c2nnc(C(=O)NCCN3CCCC3)n2-c2ccc(CN3CCOCC3)cc2)c(O)cc1O] | 5-(2,4-dihydroxy-5- isopropylphenyl)-4-(4- (morpholinomethyl)phenyl)- N-(2-(pyrrolidin-1-yl)ethyl)- 4H-1,2,4-triazole-3- carboxamide |
| 106. | 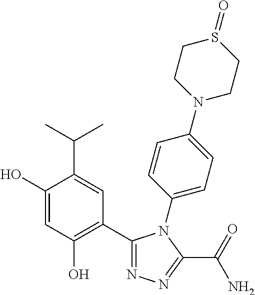 [SMILES: CC(C)c1cc(-c2nnc(C(N)=O)n2-c2ccc(N3CCS(=O)CC3)cc2)c(O)cc1O] | |
| 107. | 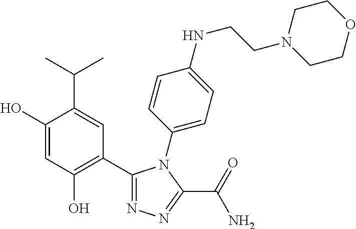 [SMILES: CC(C)c1cc(-c2nnc(C(N)=O)n2-c2ccc(NCCN3CCOCC3)cc2)c(O)cc1O] | 5-(2,4-dihydroxy-5- isopropylphenyl)-4-(4-(2- morpholinoethylamino) phenyl)-4H-1,2,4-triazole-3- carboxamide |
| 108. | 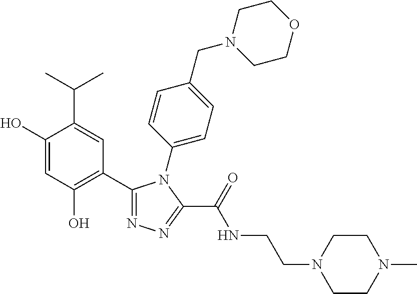 [SMILES: CC(C)c1cc(-c2nnc(C(=O)NCCN3CCN(C)CC3)n2-c2ccc(CN3CCOCC3)cc2)c(O)cc1O] | 5-(2,4-dihydroxy-5- isopropylphenyl)-N-(2-(4- methylpiperazin-1-yl)ethyl)- 4-(4- (morpholinomethyl)phenyl)- 4H-1,2,4-triazole-3- carboxamide |
| 109. | 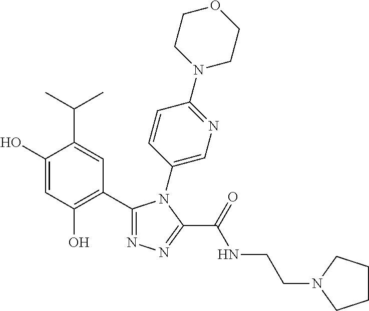 [SMILES: CC(C)c1cc(-c2nnc(C(=O)NCCN3CCCC3)n2-c2ccc(N3CCOCC3)nc2)c(O)cc1O] | 5-(2,4-dihydroxy-5- isopropylphenyl)-4-(6- morpholinopyridin-3-yl)-N- (2-(pyrrolidin-1-yl)ethyl)- 4H-1,2,4-triazole-3- carboxamide |
| 110. | 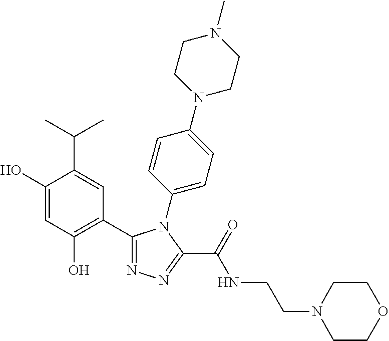 [SMILES: CC(C)c1cc(-c2nnc(C(=O)NCCN3CCOCC3)n2-c2ccc(N3CCN(C)CC3)cc2)c(O)cc1O] | 5-(2,4-dihydroxy-5- isopropylphenyl)-4-(4-(4- methylpiperazin-1- yl)phenyl)-N-(2- morpholinoethyl)-4H-1,2,4- triazole-3-carboxamide |
| 111. | 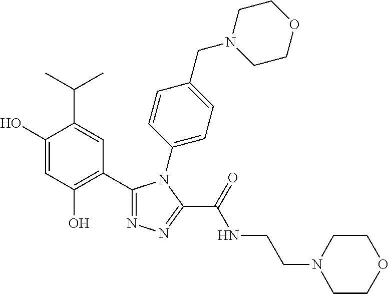 [SMILES: CC(C)c1cc(-c2nnc(C(=O)NCCN3CCOCC3)n2-c2ccc(CN3CCOCC3)cc2)c(O)cc1O] | 5-(2,4-dihydroxy-5- isopropylphenyl)-N-(2- morpholinoethyl)-4-(4- (morpholinomethyl)phenyl)- 4H-1,2,4-triazole-3- carboxamide |
| 112. | 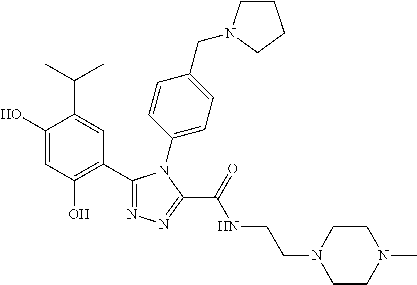 [SMILES: CC(C)c1cc(-c2nnc(C(=O)NCCN3CCN(C)CC3)n2-c2ccc(CN3CCCC3)cc2)c(O)cc1O] | 5-(2,4-dihydroxy-5- isopropylphenyl)-N-(2-(4- methylpiperazin-1-yl)ethyl)- 4-(4-(pyrrolidin-1- ylmethyl)phenyl)-4H-1,2,4- triazole-3-carboxamide |
| 113. | 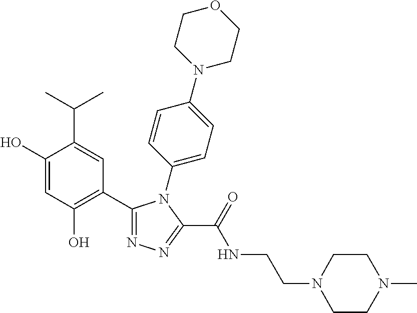 [SMILES: CC(C)c1cc(-c2nnc(C(=O)NCCN3CCN(C)CC3)n2-c2ccc(N3CCOCC3)cc2)c(O)cc1O] | 5-(2,4-dihydroxy-5- isopropylphenyl)-N-(2-(4- methylpiperazin-1-yl)ethyl)- 4-(4-morpholinophenyl)-4H- 1,2,4-triazole-3-carboxamide |
| 114. | 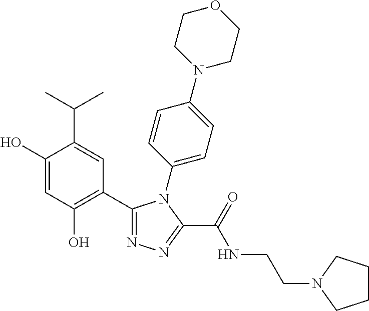 [SMILES: CC(C)c1cc(-c2nnc(C(=O)NCCN3CCCC3)n2-c2ccc(N3CCOCC3)cc2)c(O)cc1O] | 5-(2,4-dihydroxy-5- isopropylphenyl)-4-(4- morpholinophenyl)-N-(2- (pyrrolidin-1-yl)ethyl)-4H- 1,2,4-triazole-3-carboxamide |
| 115. | 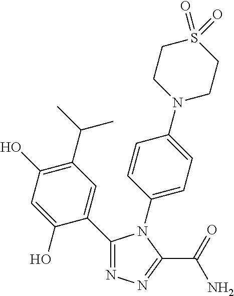 [SMILES: CC(C)c1cc(-c2nnc(C(N)=O)n2-c2ccc(N3CCS(=O)(=O)CC3)cc2)c(O)cc1O] | |
| 116. | 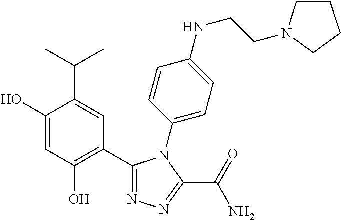 [SMILES: CC(C)c1cc(-c2nnc(C(N)=O)n2-c2ccc(NCCN3CCCC3)cc2)c(O)cc1O] | 5-(2,4-dihydroxy-5- isopropylphenyl)-4-(4-(2- (pyrrolidin-1- yl)ethylamino)phenyl)-4H- 1,2,4-triazole-3-carboxamide |
| 117. | 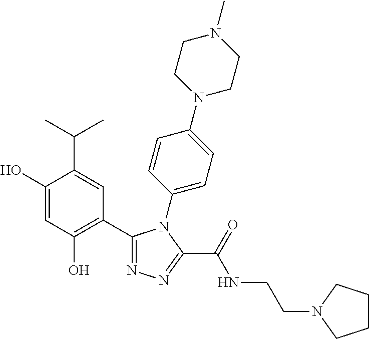 [SMILES: CC(C)c1cc(-c2nnc(C(=O)NCCN3CCCC3)n2-c2ccc(N3CCN(C)CC3)cc2)c(O)cc1O] | 5-(2,4-dihydroxy-5- isopropylphenyl)-4-(4-(4- methylpiperazin-1- yl)phenyl)-N-(2-(pyrrolidin- 1-yl)ethyl)-4H-1,2,4-triazole- 3-carboxamide |
| 118. | 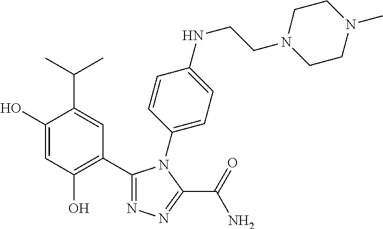 [SMILES: CC(C)c1cc(-c2nnc(C(N)=O)n2-c2ccc(NCCN3CCN(C)CC3)cc2)c(O)cc1O] | 5-(2,4-dihydroxy-5- isopropylphenyl)-4-(4-(2-(4- methylpiperazin-1- yl)ethylamino)phenyl)-4H- 1,2,4-triazole-3-carboxamide |
| 119. | 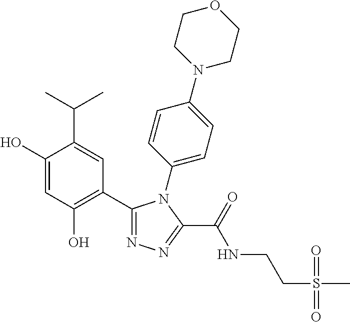 [SMILES: CC(C)c1cc(-c2nnc(C(=O)NCCS(C)(=O)=O)n2-c2ccc(N3CCOCC3)cc2)c(O)cc1O] | 5-(2,4-dihydroxy-5- isopropylphenyl)-N-(2- (methylsulfonyl)ethyl)-4-(4- morpholinophenyl)-4H- 1,2,4-triazole-3-carboxamide |
| 120. | 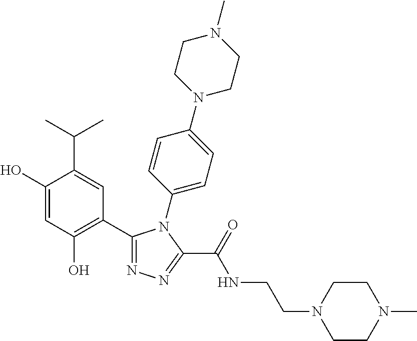 [SMILES: CC(C)c1cc(-c2nnc(C(=O)NCCN3CCN(C)CC3)n2-c2ccc(N3CCN(C)CC3)cc2)c(O)cc1O] | 5-(2,4-dihydroxy-5- isopropylphenyl)-N-(2-(4- methylpiperazin-1-yl)ethyl)- 4-(4-(4-methylpiperazin-1- yl)phenyl)-4H-1,2,4-triazole- 3-carboxamide |
| 121. | 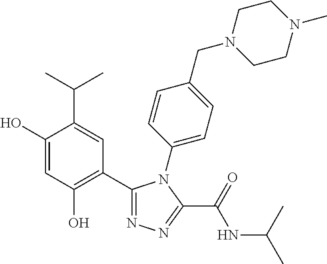 [SMILES: CC(C)NC(=O)c1nnc(-c2cc(C(C)C)c(O)cc2O)n1-c1ccc(CN2CCN(C)CC2)cc1] | 5-(2,4-dihydroxy-5- isopropylphenyl)-N- isopropyl-4-(4-((4- methylpiperazin-1- yl)methyl)phenyl)-4H-1,2,4- triazole-3-carboxamide |
| 122. | 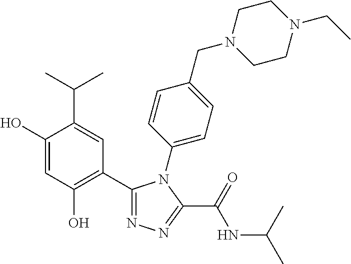 [SMILES: CCN1CCN(Cc2ccc(-n3c(C(=O)NC(C)C)nnc3-c3cc(C(C)C)c(O)cc3O)cc2)CC1] | 5-(2,4-dihydroxy-5- isopropylphenyl)-4-(4-((4- ethylpiperazin-1- yl)methyl)phenyl)-N- isopropyl-4H-1,2,4-triazole- 3-carboxamide |
| 123. | 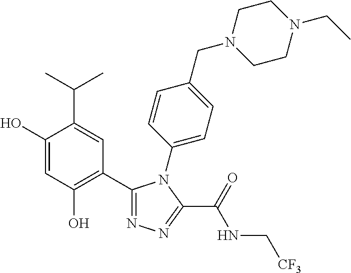 [SMILES: CCN1CCN(Cc2ccc(-n3c(C(=O)NCC(F)(F)F)nnc3-c3cc(C(C)C)c(O)cc3O)cc2)CC1] | 5-(2,4-dihydroxy-5- isopropylphenyl)-4-(4-((4- ethylpiperazin-1- yl)methyl)phenyl)-N-(2,2,2- trifluoroethyl)-4H-1,2,4- triazole-3-carboxamide |
| 124. | 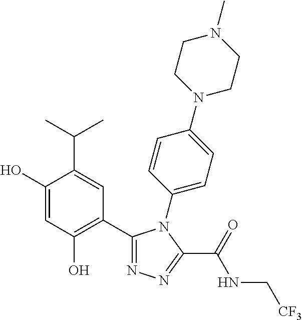 [SMILES: CC(C)c1cc(-c2nnc(C(=O)NCC(F)(F)F)n2-c2ccc(N3CCN(C)CC3)cc2)c(O)cc1O] | 5-(2,4-dihydroxy-5- isopropylphenyl)-4-(4-(4- methylpiperazin-1- yl)phenyl)-N-(2,2,2- trifluoroethyl)-4H-1,2,4- triazole-3-carboxamide |
| 125. | 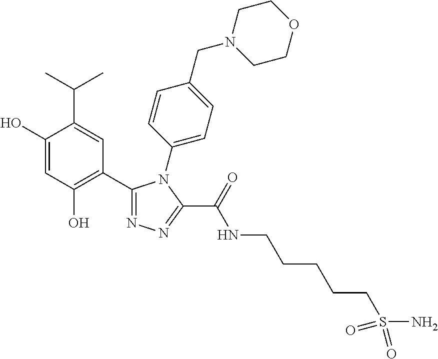 [SMILES: CC(C)c1cc(-c2nnc(C(=O)NCCCCCS(N)(=O)=O)n2-c2ccc(CN3CCOCC3)cc2)c(O)cc1O] | 5-(2,4-dihydroxy-5- isopropylphenyl)-4-(4- (morpholinomethyl)phenyl)- N-(5-sulfamoylpentyl)-4H- 1,2,4-triazole-3-carboxamide |

In an embodiment, the invention provides a method of treating polycystic kidney disease in a subject in need thereof, the method comprising administering to a subject an Hsp90 inhibitor or a pharmaceutically acceptable salt thereof. In an embodiment, the method further comprises administering one or more other therapies to the subject in need thereof (e.g., one or more therapeutic agents that are currently being used, have been used, are known to be useful or in development for use in the treatment or amelioration of polycystic kidney disease, or one or more symptoms associated with said PKD).

In an embodiment, the one or more therapeutic agents described herein can be administered sequentially or concurrently. In certain embodiments, the one or more therapeutic agents described herein improve therapeutic effect of one or more compounds described herein by functioning together with the compounds to have an additive or synergistic effect. In certain embodiments, the one or more therapeutic agents described herein reduce the side effects associated with the therapies (e.g., therapeutic agents). In certain embodiments, the one or more therapeutic agents described herein reduce the effective dosage of one or more of the therapies.

The one or more therapeutic agents described herein can be administered to a subject, preferably a human subject, in the same pharmaceutical composition. In alternative embodiments, the one or more therapeutic agents described herein can be administered concurrently to a subject in separate pharmaceutical compositions. The therapeutic agents may be administered to a subject by the same or different routes of administration.

The therapeutic agents described herein can be administered to a subject by any route known to one of skill in the art. Examples of routes of administration include, but are not limited to, parenteral, e.g., intravenous, intradermal, subcutaneous, oral (e.g., inhalation), intranasal, transdermal (topical), transmucosal, and rectal administration.

In some embodiments, the present invention also provides pharmaceutical formulations for the treatment, prophylaxis, and amelioration of polycystic kidney disease. The pharmaceutical formulations described herein are formulated to be compatible with its intended route of administration. Examples of routes of administration include parenteral, e.g., intravenous, intradermal, subcutaneous, oral, intranasal (e.g., inhalation), transdermal (topical), transmucosal, and rectal administration. In a specific embodiment, the formulation is formulated in accordance with routine procedures as a pharmaceutical composition adapted for intravenous, subcutaneous, intramuscular, oral, intranasal or topical administration to human beings. In one embodiment, the formulation is formulated in accordance with routine procedures for subcutaneous administration to human beings.

In an embodiment, the invention provides a method of treating polycystic kidney disease in a subject in need thereof, the method comprising administering to a subject an effective amount of an Hsp90 inhibitor described herein, or a pharmaceutically acceptable salt thereof. In an embodiment, the method further comprises administering one or more other therapies to the subject in need thereof. In an embodiment, the one or more other therapies include the angiotensin converting enzyme inhibitors (ACE inhibitors) such as benazepril, captopril, enalapril, fosinopril, lisinopril, moexipril, perindopril, quinapril, ramipril, and trandolapril; the angiotensin II receptor blockers (ARBs) such as candesartan, eprosartan, irbesartan, telmisartan, valsartan, losartan, and olmesartan; the arginine vasopressin (AVP) V2-receptors such as tolvaptan, lixivaptan, mozavaptan, and satavaptan; and other therapeutic agents such as SKI-606, sirolimus, and rapamycin.

In an embodiment, the invention provides a method of treating polycystic kidney disease in a subject in need thereof, the method comprising administering to a subject an effective amount of an Hsp90 inhibitor including geldanamycin derivatives, e.g., a benzoquinone or hygroquinone ansamycin HSP90 inhibitor such as IPI-493 (CAS No. 64202-81-9) and/or IPI-504 (CAS No. 857402-63-2); 17-AAG CAS No. 75747-14-7), BIIB-021 (CNF-2024, CAS No. 848695-25-0), BUB-028, AUY-922 (also known as VER-49009, CAS No. 747412-49-3), SNX-5422 (CAS No. 908115-27-5), AT-13387 (CAS No. 912999-49-6), XL-888, MPC-3100, CU-0305, 17-DMAG (CAS No. 467214-21-7), CNF-1010 (CAS No. 946090-39-7), Macbecin (e.g., Macbecin I (CAS No. 73341-72-7), Macbecin II (CAS No. 73341-73-8)), CCT-018159 (CAS No. 171009-07-7), CCT-129397 (CAS No. 940289-57-6), PU-H71 (CAS No. 873436-91-0), or PF-04928473 (SNX-2112, CAS No. 945626-71-1), 1,2,4-triazole derivatives such as ganetespib, and compound 111 or Cpd 111 (5-(2,4-dihydroxy-5-isopropylphenyl)-N-(2-morpholinoethyl)-4-(4-(morpholinomethyl)phenyl)-4H-1,2,4-triazole-3-carboxamide), or a pharmaceutically acceptable salt thereof. In an embodiment, the method further comprises administering one or more other therapies to the subject in need thereof. In an embodiment, the one or more other therapies include the angiotensin converting enzyme inhibitors (ACE inhibitors) such as benazepril, captopril, enalapril, fosinopril, lisinopril, moexipril, perindopril, quinapril, ramipril, and trandolapril; the angiotensin II receptor blockers (ARBs) such as candesartan, eprosartan, irbesartan, telmisartan, valsartan, losartan, and olmesartan; the arginine vasopressin (AVP) V2-receptors such as tolvaptan, lixivaptan, mozavaptan, and satavaptan; and other therapeutic agents such as SKI-606, sirolimus, and rapamycin.

In an embodiment, the invention provides a method of treating polycystic kidney disease in a subject in need thereof, the method comprising administering to a subject an Hsp90 inhibitor according to formulae (I)-(VII) or a compound in Table 1, or a pharmaceutically acceptable salt thereof. In an embodiment, the method further comprises administering one or more other therapies to the subject in need thereof. In an embodiment, the one or more other therapies include the angiotensin converting enzyme inhibitors (ACE inhibitors) such as benazepril, captopril, enalapril, fosinopril, lisinopril, moexipril, perindopril, quinapril, ramipril, and trandolapril; the angiotensin II receptor blockers (ARBs) such as candesartan, eprosartan, irbesartan, telmisartan, valsartan, losartan, and olmesartan; the arginine vasopressin (AVP) V2-receptors such as tolvaptan, lixivaptan, mozavaptan, and satavaptan; and other therapeutic agents such as SKI-606, sirolimus, and rapamycin.

In an embodiment, the method of treating a subject with polycystic kidney disease includes administering to the subject an effective amount of a triazolone compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof. In an embodiment, the compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof is used for treating a subject in combiantion with polycystic kidney disease in combination with an ACE inhibitor such as benazepril, captopril, enalapril, fosinopril, lisinopril, moexipril, perindopril, quinapril, ramipril, or trandolapril; and an arginine vasopressin (AVP) V2-receptor such as tolvaptan, lixivaptan, mozavaptan, or satavaptan, or other therapeutic agents such as SKI-606, sirolimus, or rapamycin. In an embodiment, the compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof is used for treating a subject in combination with lisinopril. In an embodiment, the compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof is used for treating a subject in combination with tolvaptan. In an embodiment, the compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof is used for treating a subject in combination with lisinopril and tolvaptan.

In an embodiment, the method of treating a subject with polycystic kidney disease includes administering to the subject an effective amount of a triazolone compound of 5-(2,4-dihydroxy-5-isopropylphenyl)-4-(4-(morpholinomethyl)phenyl)-N-(5-sulfamoylpentyl)-4H-1,2,4-triazole-3-carboxamide, or a pharmaceutically acceptable salt thereof. In an embodiment, the compound of 5-(2,4-dihydroxy-5-isopropylphenyl)-4-(4-(morpholinomethyl)phenyl)-N-(5-sulfamoylpentyl)-4H-1,2,4-triazole-3-carboxamide, or a pharmaceutically acceptable salt thereof is used for treating a subject with lisinopril. In an embodiment, the compound of 5-(2,4-dihydroxy-5-isopropylphenyl)-4-(4-(morpholinomethyl)phenyl)-N-(5-sulfamoylpentyl)-4H-1,2,4-triazole-3-carboxamide, or a pharmaceutically acceptable salt thereof is used for treating a subject with polycystic kidney disease in combination with an ACE inhibitor such as benazepril, captopril, enalapril, fosinopril, lisinopril, moexipril, perindopril, quinapril, ramipril, or trandolapril; and an arginine vasopressin (AVP) V2-receptor such as tolvaptan, lixivaptan, mozavaptan, or satavaptan; or other therapeutic agents such as SKI-606, sirolimus, or rapamycin. In an embodiment, the compound of 5-(2,4-dihydroxy-5-isopropylphenyl)-4-(4-(morpholinomethyl)phenyl)-N-(5-sulfamoylpentyl)-4H-1,2,4-triazole-3-carboxamide, or a pharmaceutically acceptable salt thereof is used for treating a subject in combination with lisinopril. In an embodiment, the compound of 5-(2,4-dihydroxy-5-isopropylphenyl)-4-(4-(morpholinomethyl)phenyl)-N-(5-sulfamoylpentyl)-4H-1,2,4-triazole-3-carboxamide, or a pharmaceutically acceptable salt thereof is used for treating a subject in combination with tolvaptan. In an embodiment, the compound of 5-(2,4-dihydroxy-5-isopropylphenyl)-4-(4-(morpholinomethyl)phenyl)-N-(5-sulfamoylpentyl)-4H-1,2,4-triazole-3-carboxamide, or a pharmaceutically acceptable salt thereof is used for treating a subject in combination with lisinopril and tolvaptan.

In an embodiment, the method of treating a subject with polycystic kidney disease includes administering to the subject an effective amount of a triazolone compound of 5-(2,4-dihydroxy-5-isopropylphenyl)-N-(2-morpholinoethyl)-4-(4-morpholinophenyl)-4H-1,2,4-triazole-3-carboxamide, or a pharmaceutically acceptable salt thereof. In an embodiment, the compound of 5-(2,4-dihydroxy-5-isopropylphenyl)-N-(2-morpholinoethyl)-4-(4-morpholinophenyl)-4H-1,2,4-triazole-3-carboxamide, or a pharmaceutically acceptable salt thereof is used for treating a subject with polycystic kidney disease in combination with an ACE inhibitor such as benazepril, captopril, enalapril, fosinopril, lisinopril, moexipril, perindopril, quinapril, ramipril, or trandolapril; and an arginine vasopressin (AVP) V2-receptor such as tolvaptan, lixivaptan, mozavaptan, or satavaptan; or other therapeutic agents such as SKI-606, sirolimus, or rapamycin. In an embodiment, the compound of 5-(2,4-dihydroxy-5-isopropylphenyl)-N-(2-morpholinoethyl)-4-(4-morpholinophenyl)-4H-1,2,4-triazole-3-carboxamide, or a pharmaceutically acceptable salt thereof is used for treating a subject in combination with lisinopril. In an embodiment, the compound of 5-(2,4-dihydroxy-5-isopropylphenyl)-N-(2-morpholinoethyl)-4-(4-morpholinophenyl)-4H-1,2,4-triazole-3-carboxamide, or a pharmaceutically acceptable salt thereof is used for treating a subject in combination with tolvaptan. In an embodiment, the compound of 5-(2,4-dihydroxy-5-isopropylphenyl)-N-(2-morpholinoethyl)-4-(4-morpholinophenyl)-4H-1,2,4-triazole-3-carboxamide, or a pharmaceutically acceptable salt thereof is used for treating a subject in combination with lisinopril and tolvaptan.

In an embodiment, the method of treating a subject with polycystic kidney disease includes administering to the subject an effective amount of a triazolone compound of 5-(2,4-dihydroxy-5-isopropylphenyl)-4-(4-(morpholinomethyl)phenyl)-N-(2-(pyrrolidin-1-yl)ethyl)-4H-1,2,4-triazole-3-carboxamide, or a pharmaceutically acceptable salt thereof. In an embodiment, the compound of 5-(2,4-dihydroxy-5-isopropylphenyl)-4-(4-(morpholinomethyl)phenyl)-N-(2-(pyrrolidin-1-yl)ethyl)-4H-1,2,4-triazole-3-carboxamide, or a pharmaceutically acceptable salt thereof is used for treating a subject with polycystic kidney disease in combination with an ACE inhibitor such as benazepril, captopril, enalapril, fosinopril, lisinopril, moexipril, perindopril, quinapril, ramipril, or trandolapril; and an arginine vasopressin (AVP) V2-receptor such as tolvaptan, lixivaptan, mozavaptan, or satavaptan; or other therapeutic agents such as SKI-606, sirolimus, or rapamycin. In an embodiment, the compound of 5-(2,4-dihydroxy-5-isopropylphenyl)-4-(4-(morpholinomethyl)phenyl)-N-(2-(pyrrolidin-1-yl)ethyl)-4H-1,2,4-triazole-3-carboxamide, or a pharmaceutically acceptable salt thereof is used for treating a subject in combination with lisinopril. In an embodiment, the compound of 5-(2,4-dihydroxy-5-isopropylphenyl)-4-(4-(morpholinomethyl)phenyl)-N-(2-(pyrrolidin-1-yl)ethyl)-4H-1,2,4-triazole-3-carboxamide, or a pharmaceutically acceptable salt thereof is used for treating a subject in combination with tolvaptan. In an embodiment, the compound of 5-(2,4-dihydroxy-5-isopropylphenyl)-4-(4-(morpholinomethyl)phenyl)-N-(2-(pyrrolidin-1-yl)ethyl)-4H-1,2,4-triazole-3-carboxamide, or a pharmaceutically acceptable salt thereof is used for treating a subject in combination with lisinopril and tolvaptan.

In an embodiment, the method of treating a subject with polycystic kidney disease includes administering to the subject an effective amount of a triazolone compound of 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide, or a pharmaceutically acceptable salt thereof. In an embodiment, the compound of 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide, or a pharmaceutically acceptable salt thereof is used for treating a subject with polycystic kidney disease in combination with an ACE inhibitor such as benazepril, captopril, enalapril, fosinopril, lisinopril, moexipril, perindopril, quinapril, ramipril, or trandolapril; and an arginine vasopressin (AVP) V2-receptor such as tolvaptan, lixivaptan, mozavaptan, or satavaptan; or other therapeutic agents such as SKI-606, sirolimus, or rapamycin. In an embodiment, the compound of 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide, or a pharmaceutically acceptable salt thereof is used for treating a subject in combination with lisinopril. In an embodiment, the compound of 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide, or a pharmaceutically acceptable salt thereof is used for treating a subject in combination with tolvaptan. In an embodiment, the compound of 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide, or a pharmaceutically acceptable salt thereof is used for treating a subject in combination with lisinopril and tolvaptan.

In an embodiment, the method of treating a subject with polycystic kidney disease includes administering to the subject an effective amount of a triazolone compound of 5-(2,4-dihydroxy-5-isopropylphenyl)-4-(4-((4-ethylpiperazin-1-yl)methyl)phenyl)-N-(2,2,2-trifluoroethyl)-4H-1,2,4-triazole-3-carboxamide, or a pharmaceutically acceptable salt thereof. In an embodiment, the compound of 5-(2,4-dihydroxy-5-isopropylphenyl)-4-(4-((4-ethylpiperazin-1-yl)methyl)phenyl)-N-(2,2,2-trifluoroethyl)-4H-1,2,4-triazole-3-carboxamide, or a pharmaceutically acceptable salt thereof is used for treating a subject with polycystic kidney disease in combination with an ACE inhibitor such as benazepril, captopril, enalapril, fosinopril, lisinopril, moexipril, perindopril, quinapril, ramipril, or trandolapril; an arginine vasopressin (AVP) V2-receptor such as tolvaptan, lixivaptan, mozavaptan, or satavaptan, and other therapeutic agents such as SKI-606, sirolimus, or rapamycin. In an embodiment, the compound of 5-(2,4-dihydroxy-5-isopropylphenyl)-4-(4-((4-ethylpiperazin-1-yl)methyl)phenyl)-N-(2,2,2-trifluoroethyl)-4H-1,2,4-triazole-3-carboxamide, or a pharmaceutically acceptable salt thereof is used for treating a subject in combination with lisinopril. In an embodiment, the compound of 5-(2,4-dihydroxy-5-isopropylphenyl)-4-(4-((4-ethylpiperazin-1-yl)methyl)phenyl)-N-(2,2,2-trifluoroethyl)-4H-1,2,4-triazole-3-carboxamide, or a pharmaceutically acceptable salt thereof is used for treating a subject in combination with tolvaptan. In an embodiment, the compound of 5-(2,4-dihydroxy-5-isopropylphenyl)-4-(4-((4-ethylpiperazin-1-yl)methyl)phenyl)-N-(2,2,2-trifluoroethyl)-4H-1,2,4-triazole-3-carboxamide, or a pharmaceutically acceptable salt thereof is used for treating a subject in combination with lisinopril and tolvaptan.

In general, the recommended daily dose range of a triazolone compound for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different cancers, as will be readily known by those of ordinary skill in the art. Similarly, amounts sufficient to prevent, manage, treat or ameliorate such cancers, but insufficient to cause, or sufficient to reduce, adverse effects associated with the triazolone compounds described herein are also encompassed by the above described dosage amounts and dose frequency schedules. Further, when a patient is administered multiple dosages of a triazolone compound described herein, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition comprising a triazolone compound described herein administered to prevent, treat, manage, or ameliorate polycystic kidney disease, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition comprising a compound described herein administered to prevent, treat, manage, or ameliorate polycystic kidney disease, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 200 mg, 1 mg to 175 mg, 1 mg to 150 mg, 1 mg to 125 mg, 1 mg to 100 mg, 1 mg to 75 mg, 1 mg to 50 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 or 7 days, or once weekly, once every two weeks, once every three weeks or once monthly.

In certain embodiments, one or more compounds described herein and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In a specific embodiment, the method includes preventing, treating, managing, or ameliorating polycystic kidney disease, or one or more symptoms thereof, comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

In an embodiment, the invention also provides the use of at least one of these Hsp90 inhibitory compounds or a pharmaceutically acceptable salt thereof for the manufacture of a medicament for the treatment of a subject with polycystic kidney disease. In an embodiment, the invention further provides the use of at least one of these Hsp90 inhibitory compounds or a pharmaceutically acceptable salt thereof for the manufacture of a medicament for the treatment of a subject with polycystic kidney disease in combination with one or more of benazepril, captopril, enalapril, fosinopril, lisinopril, moexipril, perindopril, quinapril, ramipril, trandolapril, candesartan, eprosartan, irbesartan, telmisartan, valsartan, losartan, olmesartan, tolvaptan, lixivaptan, mozavaptan, SKI-606, sirolimus, rapamycin, and satavaptan.

In an embodiment, the invention also provides the use of at least one of these Hsp90 inhibitory compounds described herein or a pharmaceutically acceptable salt thereof for the manufacture of a medicament for the treatment of a subject with polycystic kidney disease. In an embodiment, the invention further provides the use of at least one of these Hsp90 inhibitory compounds described herein or a pharmaceutically acceptable salt thereof for the manufacture of a medicament for the treatment of a subject with polycystic kidney disease in combination with one or more of benazepril, captopril, enalapril, fosinopril, lisinopril, moexipril, perindopril, quinapril, ramipril, trandolapril, candesartan, eprosartan, irbesartan, telmisartan, valsartan, losartan, olmesartan, tolvaptan, lixivaptan, mozavaptan, SKI-606, sirolimus, rapamycin, and satavaptan.

In an embodiment, the invention also provides the use of at least one of these Hsp90 inhibitory compounds including geldanamycin derivatives, e.g., a benzoquinone or hygroquinone ansamycin HSP90 inhibitor such as IPI-493 (CAS No. 64202-81-9) and/or IPI-504 (CAS No. 857402-63-2); 17-AAG CAS No. 75747-14-7), BIIB-021 (CNF-2024, CAS No. 848695-25-0), BUB-028, AUY-922 (also known as VER-49009, CAS No. 747412-49-3), SNX-5422 (CAS No. 908115-27-5), AT-13387 (CAS No. 912999-49-6), XL-888, MPC-3100, CU-0305, 17-DMAG (CAS No. 467214-21-7), CNF-1010 (CAS No. 946090-39-7), Macbecin (e.g., Macbecin I (CAS No. 73341-72-7), Macbecin II (CAS No. 73341-73-8)), CCT-018159 (CAS No. 171009-07-7), CCT-129397 (CAS No. 940289-57-6), PU-H71 (CAS No. 873436-91-0), or PF-04928473 (SNX-2112, CAS No. 945626-71-1), 1,2,4-triazole derivatives such as ganetespib, and compound 111 or Cpd 111 (5-(2,4-dihydroxy-5-isopropylphenyl)-N-(2-morpholinoethyl)-4-(4-(morpholinomethyl)phenyl)-4H-1,2,4-triazole-3-carboxamide), or a pharmaceutically acceptable salt thereof for the manufacture of a medicament for the treatment of a subject with polycystic kidney disease. In an embodiment, the invention further provides the use of at least one of these Hsp90 inhibitory compounds including geldanamycin derivatives, e.g., a benzoquinone or hygroquinone ansamycin HSP90 inhibitor such as IPI-493 (CAS No. 64202-81-9) and/or IPI-504 (CAS No. 857402-63-2); 17-AAG CAS No. 75747-14-7), BIIB-021 (CNF-2024, CAS No. 848695-25-0), BUB-028, AUY-922 (also known as VER-49009, CAS No. 747412-49-3), SNX-5422 (CAS No. 908115-27-5), AT-13387 (CAS No. 912999-49-6), XL-888, MPC-3100, CU-0305, 17-DMAG (CAS No. 467214-21-7), CNF-1010 (CAS No. 946090-39-7), Macbecin (e.g., Macbecin I (CAS No. 73341-72-7), Macbecin II (CAS No. 73341-73-8)), CCT-018159 (CAS No. 171009-07-7), CCT-129397 (CAS No. 940289-57-6), PU-H71 (CAS No. 873436-91-0), or PF-04928473 (SNX-2112, CAS No. 945626-71-1), 1,2,4-triazole derivatives such as ganetespib, and compound 111 or Cpd 111 (5-(2,4-dihydroxy-5-isopropylphenyl)-N-(2-morpholinoethyl)-4-(4-(morpholinomethyl)phenyl)-4H-1,2,4-triazole-3-carboxamide), or a pharmaceutically acceptable salt thereof for the manufacture of a medicament for the treatment of a subject with polycystic kidney disease in combination with one or more of benazepril, captopril, enalapril, fosinopril, lisinopril, moexipril, perindopril, quinapril, ramipril, trandolapril, candesartan, eprosartan, irbesartan, telmisartan, valsartan, losartan, olmesartan, tolvaptan, lixivaptan, mozavaptan, SKI-606, sirolimus, rapamycin, and satavaptan.

In an embodiment, the invention also provides the use of an Hsp90 inhibitor according to formulae (I)-(VII) or a compound in Table 1, or a pharmaceutically acceptable salt thereof for the manufacture of a medicament for the treatment of a subject with polycystic kidney disease. In an embodiment, the invention further provides the use of an Hsp90 inhibitor according to formulae (I)-(VII) or a compound in Table 1, or a pharmaceutically acceptable salt thereof for the manufacture of a medicament for the treatment of a subject with polycystic kidney disease in combination with one or more of benazepril, captopril, enalapril, fosinopril, lisinopril, moexipril, perindopril, quinapril, ramipril, trandolapril, candesartan, eprosartan, irbesartan, telmisartan, valsartan, losartan, olmesartan, tolvaptan, lixivaptan, mozavaptan, SKI-606, sirolimus, rapamycin, and satavaptan.

In an embodiment, the invention also provides the use of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for the manufacture of a medicament for the treatment of a subject with polycystic kidney disease. In an embodiment, the invention further provides the use of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for the manufacture of a medicament for the treatment of a subject with polycystic kidney disease in combination with one or more of benazepril, captopril, enalapril, fosinopril, lisinopril, moexipril, perindopril, quinapril, ramipril, trandolapril, candesartan, eprosartan, irbesartan, telmisartan, valsartan, losartan, olmesartan, tolvaptan, lixivaptan, mozavaptan, SKI-606, sirolimus, rapamycin, and satavaptan.

In an embodiment, the invention further provides the use of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for the manufacture of a medicament for the treatment of a subject with polycystic kidney disease in combination with lisinopril. In an embodiment, the invention further provides the use of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for the manufacture of a medicament for the treatment of a subject with polycystic kidney disease in combination with tolvaptan. In an embodiment, the invention further provides the use of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for the manufacture of a medicament for the treatment of a subject with polycystic kidney disease in combination with lisinopril and tolvaptan.

In an embodiment, the invention further provides at least one of these Hsp90 inhibitory compounds or a pharmaceutically acceptable salt thereof for use in treating a subject with polycystic kidney disease. In an embodiment, the invention also provides at least one of these Hsp90 inhibitory compounds or a pharmaceutically acceptable salt thereof for use in treating a subject with polycystic kidney disease in combination with one or more of benazepril, captopril, enalapril, fosinopril, lisinopril, moexipril, perindopril, quinapril, ramipril, trandolapril, candesartan, eprosartan, irbesartan, telmisartan, valsartan, losartan, olmesartan, tolvaptan, lixivaptan, mozavaptan, SKI-606, sirolimus, rapamycin, and satavaptan.

In an embodiment, the invention further provides a compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for use in treating a subject with polycystic kidney disease. In an embodiment, the invention also provides a compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for use in treating a subject with polycystic kidney disease in combination with one or more of benazepril, captopril, enalapril, fosinopril, lisinopril, moexipril, perindopril, quinapril, ramipril, trandolapril, candesartan, eprosartan, irbesartan, telmisartan, valsartan, losartan, olmesartan, tolvaptan, lixivaptan, mozavaptan, SKI-606, sirolimus, rapamycin, and satavaptan.

In an embodiment, the invention also provides a compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for use in treating a subject with polycystic kidney disease in combination with lisinopril. In an embodiment, the invention also provides a compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for use in treating a subject with polycystic kidney disease in combination with tolvaptan. In an embodiment, the invention also provides a compound of 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof for use in treating a subject with polycystic kidney disease in combination with lisinopril and tolvaptan.

### EXAMPLES

### Example 1

### Synthesis of Hsp90 Inhibitory Compounds

The Hsp90 inhibitory compounds used in the disclosed pharmaceutical compositions and methods herein can be prepared according to the procedures disclosed in U.S. Patent Publication No. 2006/0167070, and WO2009/023211.

### Example 2

### Degradation of Hsp90 Client Proteins via Inhibition of Hsp90 Activity

A. Cells and Cell Culture

Human high-Her2 breast carcinoma BT474 (HTB-20), SK-BR-3 (HTB-30) and MCF-7 breast carcinoma (HTB-22) from American Type Culture Collection, VA, USA were grown in Dulbecco's modified Eagle's medium with 4 mM L-glutamine and antibiotics (100 IU/ml penicillin and 100 ug/ml streptomycine;GibcoBRL). To obtain exponential cell growth, cells were trypsinized, counted and seeded at a cell density of 0.5×10⁶ cells/ml regularly, every 3 days. All experiments were performed on day 1 after cell passage.

B. Degradation of Her2 in Cells After Treatment with a Compound of the Invention

1. Method 1

BT-474 cells are treated with 0.5 μM, 2 μM, or 5 μM of 17AAG (a positive control) or 0.5 μM, 2 μM, or 5 μM of a compound of the invention overnight in DMEM medium. After treatment, each cytoplasmic sample is prepared from 1×10⁶ cells by incubation of cell lysis buffer (#9803, cell Signaling Technology) on ice for 10 minutes. The resulting supernatant used as the cytosol fractions is dissolved with sample buffer for SDS-PAGE and run on a SDS-PAGE gel, blotted onto a nitrocellulose membrane by using semi-dry transfer. Non-specific binding to nitrocellulose is blocked with 5% skim milk in TBS with 0.5% Tween at room temperature for 1 hour, then probed with anti-Her2/ErB2 mAb (rabbit IgG, #2242, Cell Signaling) and anti-Tubulin (T9026, Sigma) as housekeeping control protein. HRP-conjugated goat anti-rabbit IgG (H+L) and HRP-conjugated horse anti-mouse IgG (H+L) are used as secondary Ab (#7074, #7076, Cell Signaling) and LumiGLO reagent, 20× Peroxide (#7003, Cell Signaling) is used for visualization.

Her2, an Hsp90 client protein, is expected to be degraded when cells are treated with compounds of the invention. 0.5 μM of 17AAG, a known Hsp90 inhibitor which is used as a positive control, causes partial degradation of Her2.

2. Method 2

MV-4-11 cells (20,000 cells/well) were cultured in 96-well plates and maintained at 37° C. for several hours. The cells were treated with a compound of the invention or 17AAG (a positive control) at various concentrations and incubated at 37° C. for 72 hours. Cell survival was measured with Cell Counting Kit-8 (Dojindo Laboratories, Cat. #CK04).

The IC₅₀ range for Her2 degradation by compounds of the invention are listed below in Table 2.

TABLE 2
IC₅₀ range of compounds of the invention for inhibition of Hsp90

| IC₅₀ (nM) | Compound Number |
|---|---|
| ≦20 | 6, 11, 15, 28, 29, 30, 46, 63, 65, 67, 69 |
| 20 < x ≦ 50 | 1, 2, 3, 4, 5, 8, 9, 10, 12, 14, 16, 18, 20, 21, 22, 23, 24, |
| | 47, 50, 51, 52, 53, 57, 58, 60, 61, 68, 70, 71, 75, 80, |
| | 81, 82, 84, 87, 88, 89, 90, 91, 97, 108, 112, 114 |
| 50 < x < 100 | 13, 17, 54, 59, 64, 73, 77, 79, 83, 85, 95, 96, 99, 100, |
| | 102, 103, 104, 105, 107, 109, 111, 113, 115 |
| 100 < x < 500 | 19, 26, 34, 55, 62, 66, 74, 76, 78, 98, 101, 106, 110, |
| | 117 |
| 500 < x < 1000 | 86 |
| >1000 | 25, 27, 32, 33, 35, 36, 37, 38, 39, 72 |

### Example 3

### Compound 111's Inhibition of Cyst Formation and Kidney Growth in Pkd^{-/-} Mice

Mouse Strains and Drug Treatment

All experiments involving mice were approved by the Institutional Animal Care and Use Committee of Fox Chase Cancer Center. Conditional Pkd1^{-/-} mice using the Cre-flox regulatory system for targeted inactivation of the Pkd1 gene in vivo were kindly provided by Gregory Germino (National Institute of Diabetes and Digestive and Kidney Diseases). Pkd1fl/fl;Cre/Esr1+ (referred to as Pkd1^{-/-}) and control mice (Pkd1fl/fl;Cre/Esr1−; referred to as Control) mice were injected intraperitoneally with tamoxifen (250 mg/kg body weight) on postnatal day P37/P38 (+/−1 day) to induce Pkd1 deletion in the test group.

Compound 111 was formulated in 5% dextrose titrated to pH 4, and administered by tail vein injection in anesthetized mice under sterile conditions using a volume of 10 μl/g body weight. Vehicle treated mice were injected with 5% dextrose only. To investigate the activity of compound 111 on early kidney disease, mice were treated with compound 111 for 5 months, starting 2-3 days after Pkd1 deletion. Treatment began with a weekly schedule up to 4 months of age, including one week off in the 3^{rd} month of age, followed by a bi-weekly schedule from 4 to 6 month of age. 24 hours after the 16th injection, blood samples were collected and the mice were sacrificed to collect organs for histopathological and biochemical analysis. To investigate the activity of compound 111 on Late kidney disease, mice were dosed weekly with compound 111 for 10 weeks starting at 4 months of age. 24 hours after the 11^{th} dose (10 weeks of treatment), blood samples were collected and the mice were sacrificed to collect organs for histopathological and biochemical analysis.

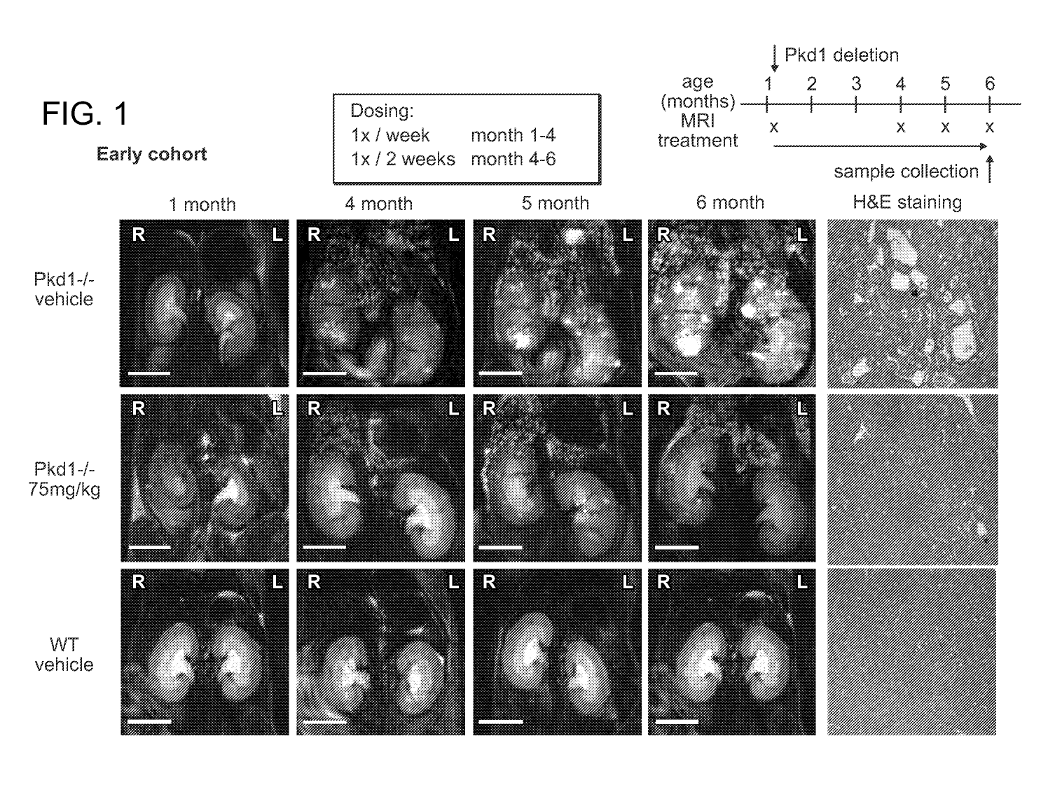

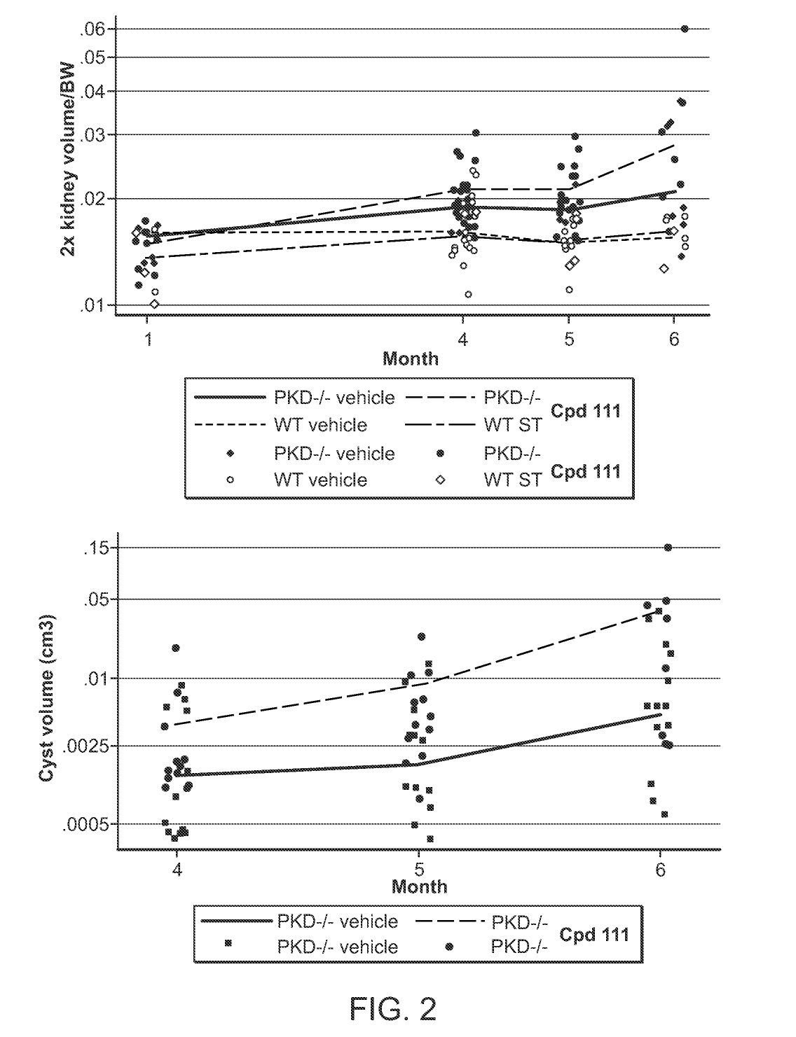

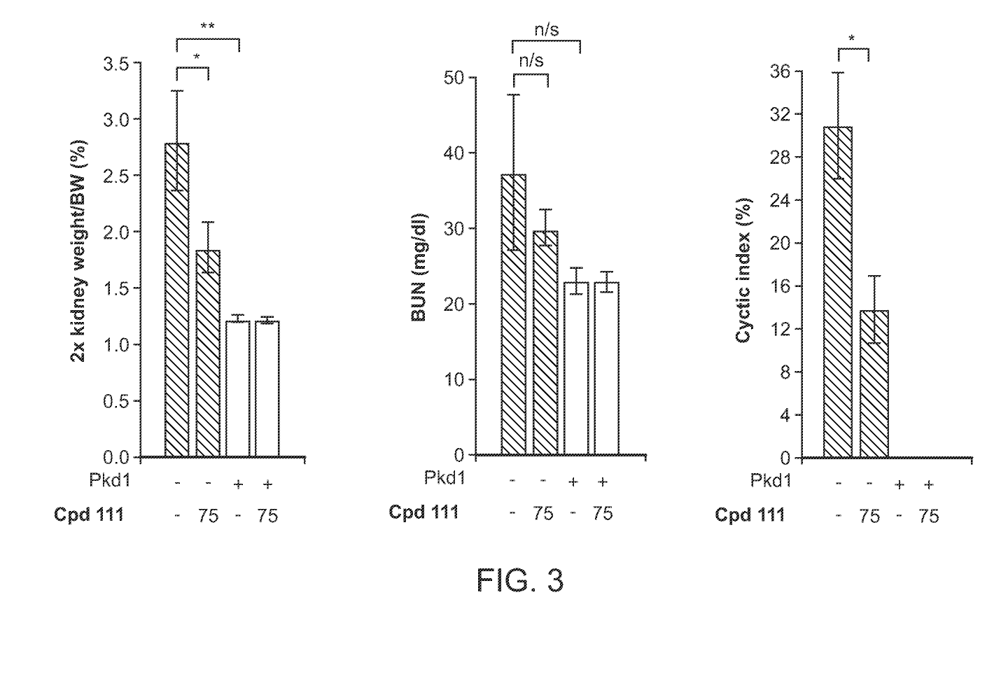

To assess the utility of HSP90 inhibition in ADPKD, Pkd1fl/fl;Cre/Esr1+ (referred to as Pkd1^{-/-}) and control Pkd1fl/fl;Cre/Esr1− (referred to as control) mice were injected with tamoxifen at 5-6 weeks of age to inactivate the Pkd1 gene. HSP90 was inhibited using compound, a resorcinolic triazole that competitively binds the N-terminal ATP pocket of HSP90. In one cohort of mice (designated Early), mice were dosed from 6 weeks-4 months of age, to assess drug effects on disease initiation (FIG. 1). Magnetic resonance imaging (MRI) assessment of the appearance of cysts and growth in kidney volume coupled with direct measurement of kidney weight and immunohistochemical assessment of cyst index indicated that 75 mg/kg of compound 111 very significantly reduced formation of renal cysts and kidney growth. Analysis of blood drawn at time of euthanasia indicated showed a trend towards reduction of blood urea nitrogen (BUN), a measure of renal function, although this did not achieve statistical significance. (FIGS. 2-3)

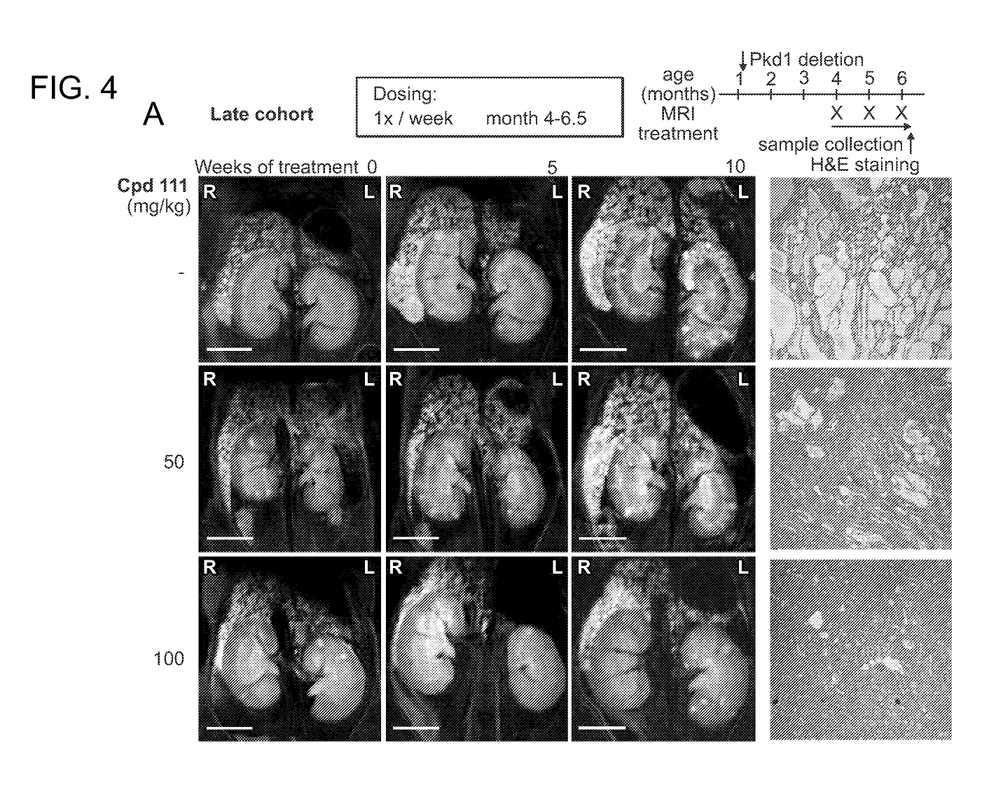

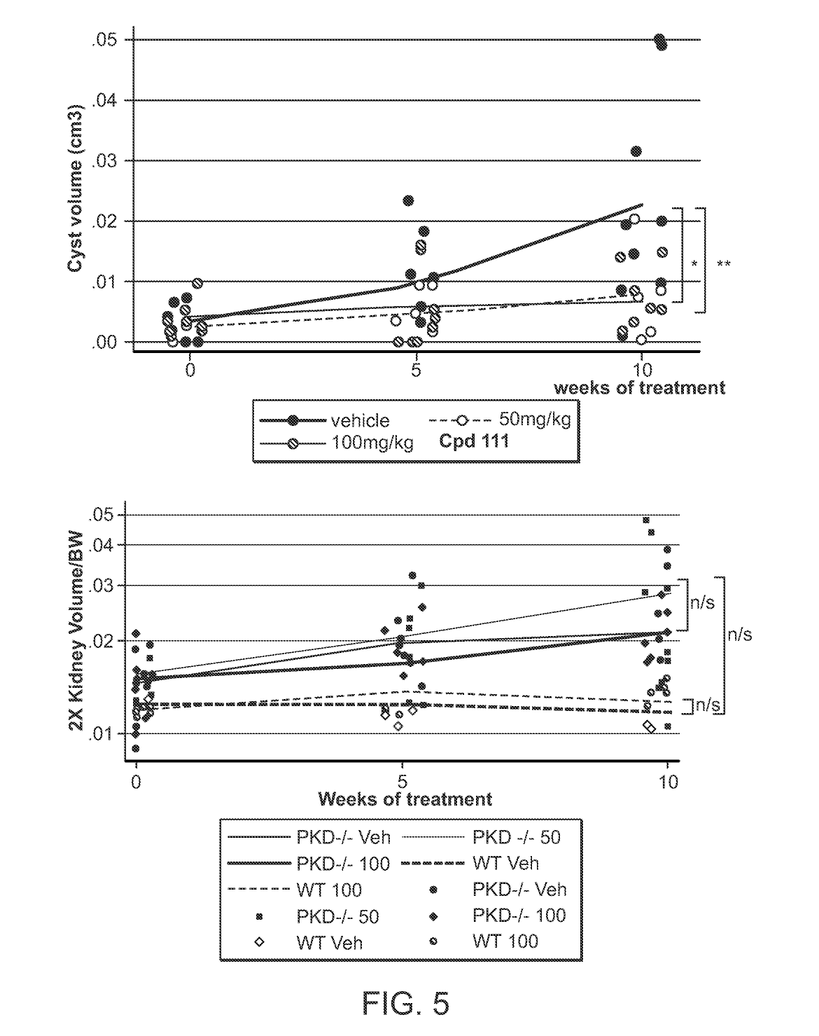

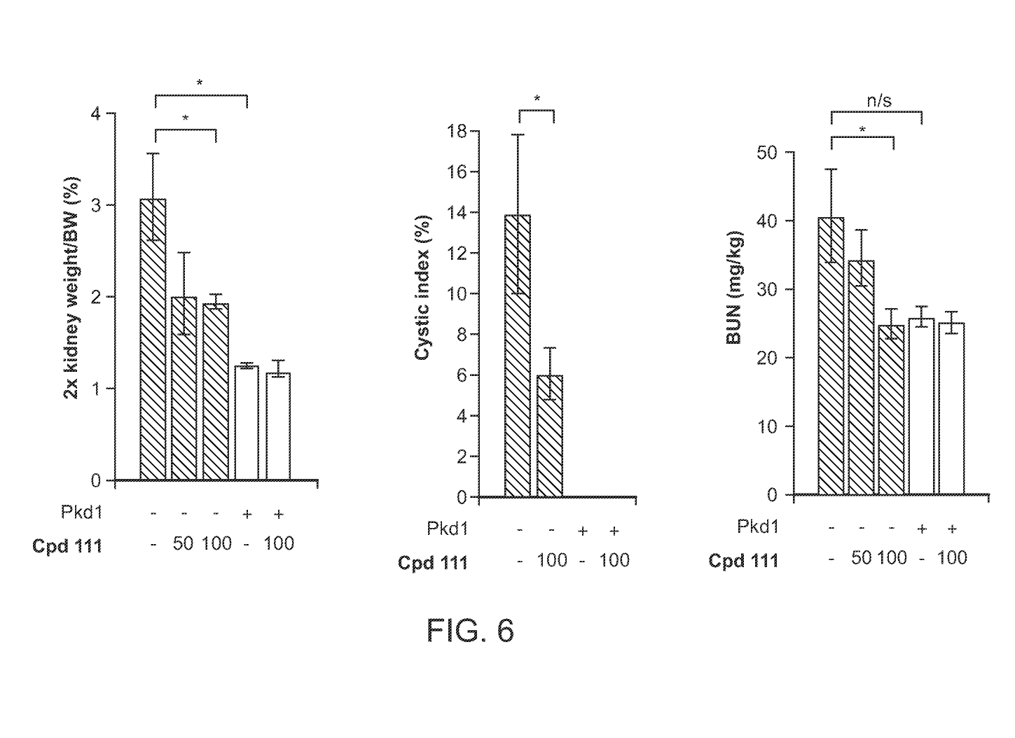

In a second cohort (designated Late), mice were dosed from 4-6 months of age, to assess drug effects on mice with Late cysts, in this case using doses of 50 mg/kg and 100 mg/kg to explore dose range (FIGS. 4-6). As with the Early cohort, a significant, dose-dependent effect of compound 111 was observed in reduction of cyst volume and kidney growth, while 100 mg/kg reduced BUN scores to normal levels.

MRI Performance and Image Analysis

Mice were imaged in a vertical bore MR system at a field strength of 7 Tesla, using a Bruker DRX300 spectrometer, Paravision 3.0.2 software, and a single tuned ¹H cylindrical radiofrequency coil. Following acquisition of a low-resolution scout scan in the axial plane, a coronal scan was set to cover the entire volume of both kidneys. A RARE (rapid acquisition of re-focused echoes) pulse sequence was employed, as it has previously been shown that RARE sequences in preclinical models of polycystic kidney disease that cysts exhibit high intensity in the MR images, and are easily distinguishable from the surrounding tissues. The acquisition parameters for the RARE scan were: echo time=17.6 msec, rare factor=8, effective echo time=73.6 msec, repetition time=4500 msec, averages=4, slice thickness=0.75 mm, field of view=2.56 mm, in-plane resolution=0.1 mm, number of slices=28. The total acquisition time was 10 minutes, 3 seconds, which was well tolerated by the mice. During the imaging procedure mice were anesthetized with 1-2% isoflurane in O₂. Images show the size and texture of the complete kidneys and at least of major parts of the liver.

Using NIH ImageJ, an open source image analysis program, kidney and cyst volume were quantified. The kidney volume estimation technique was performed as previously described by manually surrounding kidney parenchyma excluding the renal pelvis and summing up the products of area measurements of contiguous images and slice thickness. A semiautomatic threshold approach was performed for cyst volume estimation. Subsequently isolated kidney areas were prepared using defined settings for background subtraction (rolling ball radius: 20 pixels) and band passing (FFT band pass filter with structures 3-40 pixels). The threshold was set for each kidney based on the original images by targeting threshold values designating the transition between parenchyma and cyst that could be detected at the border of larger cysts in the kidneys.

Tissue Preparation, Histology, Immunohistochemical Analysis, Cystic Index, and Blood Urea Nitrogen (BUN)

Tissues were collected and fixed in 10% phosphate-buffered formaldehyde (formalin) 24-48 hrs, dehydrated and embedded in paraffin. Hematoxylin and eosin (H&E) stained sections were used for morphological evaluation purposes and unstained sections for immunohistochemical (IHC) studies.

Immunohistochemical staining was performed on 5-μm formalin fixed paraffin embedded sections. After deparaffinization and rehydration, sections were subjected to heat-induced epitope retrieval by steaming in 0.01 M citrate buffer (pH 6.0) or EDTA (pH 9.0) for 20 minutes. After quenching endogenous peroxidase activity with 3% hydrogen peroxide for 20-30 min and blocking nonspecific protein binding with goat serum, sections were incubated overnight with primary monoclonal antibodies anti-Ki67(Rat anti-mouse, Dako, 1:100) and anti-cleaved caspase 3 (Cell Signaling) at 4° C., followed by biotinylated goat anti-Rat IgG (DAKO, 1:200) for 30 min, detecting the antibody complexes with the labeled streptavidin-biotin system (DAKO), and visualizing them with the chromogen 3,3′-diaminobenzidine.

For other proteins, in order to minimize the endogenous biotin background immonodetection was performed with the Dako Envision+polymer system. Primary antibodies include anti-phospho-S6 (pSer235/p236) (Rabbit, Cell signaling, 1:1000), anti-phospho-Akt (pThr308) (Rabbit, Cell Signaling, #4060,1:50), anti-phospho-Erk1/2 (pThr202/pTyr204) (Rabbit, Cell Signaling, #4370, 1:400), anti-phosho-Aurora A (pT288)(Rabbit, Bethyl, #IHC-00067, 1:300).

The slides were then washed, counterstained with hematoxylin, dehydrated with alcohol, cleared in xylene, and mounted. Mice samples that were shown previously to express high levels of proteins were used as positive controls. The negative control was performed by replacing the primary antibody with normal rabbit IgG.

Immunostained slides were scanned using Aperio ScanScope CS scanner (Aperio, Vista, Calif.). Scanned images were then viewed with Aperio's image viewer software (ImageScope). Selected regions of interest were outlined manually by a pathologist (Cai KQ). Expression levels of pAKT, pS6, and HSP90 were measured using Aperio Positive Pixel Count V9 algorithm and proliferative index (ki-67) were quantified using Aperio Nuclear V9 algorithm.

For cystic index analysis, a grid was placed over representative images of hematoxylin-eosi-stained kidney sections, and the cystic index was calculated as the percentage of grid intersection points that bisected cystic or non-cystic areas, as described previously.

Primary Kidney Cells, Cell Culture and Drug Treatment

Primary epithelial kidney cells were derived from Pkd1−/− and control mice (from 3 independent animals per genotype) and were maintained in low calcium media containing 5% chelated horse serum. These populations were not immortalized, to avoid oncogene-associated changes in signaling pathways, and only cells between passages 3-8 were used for experiments. Cell viability was quantified using the alamarBlue® Assay 72 hours after the indicated drug treatment.

Protein Expression Analysis Using Reverse Phase Protein Arrays (RPPAs) and Western Blotting

Cells were treated with 250 μM of compound 111 or ganetespib for 24 h, then harvested and lysed for analysis by Western blot or reverse phase protein array (RPPA, performed by MD Anderson's RPPA Core Facility). Insert extended section about RPPAs. Data were visualized using the MultiExperiment Viewer (MeV) program. Hierarchical clustering (using the parameters shown above) was applied to the set of data normalized to the average of three vehicle-treated control primary cells.

Cells were lysed and resolved by SDS-Page To analyze the expression levels of individual proteins. Western Blotting was performed using standard procedures, and developed by chemiluminescence using Super Signal West Pico and Femto substrate (Thermo Fisher Scientific). Primary antibodies included anti-STAT3 (Cell Signaling, #9139), anti-phospho STAT3 (Cell Signaling, #9145), anti-Aurora A (BD, #610939), anti-HSP70 (Enzo, #ADI-SPA-820), anti-HSP90 (Enzo, #ADI-SPA-835), anti-phospho-ERK Thr202/204 (Cell Signaling, #9101), anti-CDK1 (Santa Cruz, #sc-54), anti-phospho-CDK1 Thr15 (Cell Signaling, #9111), and mouse anti-β-actin conjugated to HRP (Abcam, #ab49900). Secondary anti-mouse and anti-rabbit HRP-conjugated antibodies (GE Healthcare) were used at a dilution of 1:10,000. Quantification of signals on Western blots was done using the NIH ImageJ Imaging and Processing Analysis Software with signaling intensity normalized to β-actin.

In conclusion, inhibition of Hsp90, in a mouse model of ADPKD, demonstrates encouraging signs of activity, both in the reduction of cyst number and volume as well as improved renal function. With weekly dosing schedule being well tolerated and effective over a long period of time, Hsp90 inhibition may be suitable for long-term treatment of ADPKD patients.

### Example 4

### Combination of Compound 111 with Tolvaptan, Lisinopril, and Other Inhibitors

The study design is straightforward. Effective dosing conditions (reflected by significant biological effect and no toxicity) have been well established for mice with lisinopril and for tolvaptan, with administration beginning in animals as young as 3 weeks old. Lisinopril is provided in drinking water at 100 ug/ml. Tolvaptan is provided in ground rodent chow at 0.1% concentration. Focus is primarily on the Pkd1^{cond} model, with cystic growth induced by tamoxifen injection at P35, and treatment with agents beginning at 2 months of age, and continuing to 8 months of age. Treatment cohorts include 1) vehicle; 2) compound 111; 3) vehicle+lisinopril; 4) vehicle+tolvaptan; 5) compound 111+lisinopril; 6) compound 111+tolvaptan; 7) vehicle+lisinopril+tolvaptan; and 8) Scompound 111 lisinopril+tolvaptan (total, 160 mice). All measurements (MRI, histopathology, blood analysis) are performed as for in Example 3. Generalized linear models are to be used to assess the effect of the inhibitors. Appropriate interactions are to be included in the models to examine whether the effects of compound 111 with other inhibitors are additive or synergistic. In addition, two additional categories of measurement as controls are conducted to confirm that an effective concentration of lisinopril and tolvaptan are used, based on their mode of action. As one assay particularly relevant to lisinopril, a standard tail cuff assay is used to assess blood pressure in all treatment groups.

The combination of compound with lisinopril and/or tolvaptan results in improved functionality in limiting cyst growth. All three agents are well tolerated, so that no severe adverse effects are expected. If the results of the described study indicate exceptional control of cysts by any particular combination, a followup experiment would be to undertake extended treatment of a new cohort of animals with the treatment versus a relevant control group, to determine the maximum duration of cyst control and maintenance on treatment.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

What is claimed is:

1. A method of treating or inhibiting polycystic kidney disease in a subject, comprising administering to the subject an effective amount of an HSP90 inhibitor or a pharmaceutically acceptable carrier.

2. A method of treating or inhibiting polycystic kidney disease in a subject, comprising administering to the subject an effective amount of a compound according to the following formulae (I): 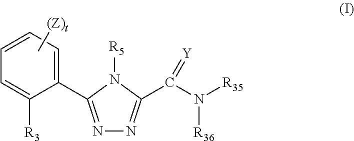
or a pharmaceutically acceptable salt thereof, wherein:
Y is O or S;
R₃ is —OH, —SH, —NR₇H, —OR₂₆, —SR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂;
R₅ is —H, —X₂₀R₅₀, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₇ and R₈, for each occurrence, is independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₂₆ is a lower alkyl;
R₃₅ and R₃₆, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl, or R₃₅ and R₃₆, together with N to which they are attached form a 5 to 7 membered heterocyclic ring;
R₅₀ is an optionally substituted aryl or an optionally substituted heteroaryl;
X₂₀ is a C1-C4 alkyl, NR_{S}, C(O), C(S), C(NR₈), or S(O)ₚ;
Z is a substituent, which is independently —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
t is 0,1,2,3, or 4;
p, for each occurrence, is independently, 1 or 2;
or a tautomer, pharmaceutically acceptable salt thereof.

3. The method of claim 2, wherein the compound is according to the following formula (II): 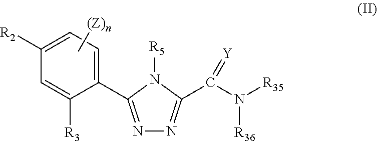
or a pharmaceutically acceptable salt thereof, wherein:
R₂ is —OH, —SH, —NR₇H, —OR₂₆, —SR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —S(O)ₚOR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, —NR₇C(NR₈)NR₁₀R₁₁, —OP(O)(OR₇)₂, or —SP(O)(OR₇)₂; and
n is 0, 1, 2, or 3.

4. The method of claim 2, wherein the compound is according to the following formula (III): 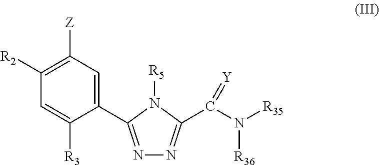
or a pharmaceutically acceptable salt thereof.

5. The method of claim 2, wherein the compound is according to the following formula (IV): 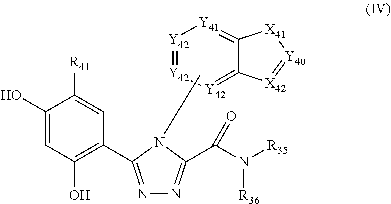
or a pharmaceutically acceptable salt thereof, wherein:
X₄₁ is O, S, or NR₄₂;
X₄₂ is CR₄₄ or N;
Y₄₀ is N or CR₄₃;
Y₄₁ is N or CR₄₅;
Y_{42,} for each occurrence, is independently N, C or CR₄₆;
R₄₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₄₂ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄₃ and R₄₄ are, independently, —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —C(O)R₇, —C(O)OR₇, —OC(O)R₇, —C(O)NR₁₀R₁₁, —NR₈C(O)R₇, —SR_{S}, —S(O)ₚR₇, —OS(O)ₚR₇, —S(O)ₚOR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;
R₄₅ is —H, —OH, —SH, —NR₇H, —OR₂₆, —SR₂₆, —NHR₂₆, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁; and
R₄₆, for each occurrence, is independently, selected from the group consisting of H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, —NR₁₀R₁₁, —OR₇, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, or —S(O)ₚNR₁₀R₁₁.

6. The method of claim 5, wherein the compound is according to the following formula (V): 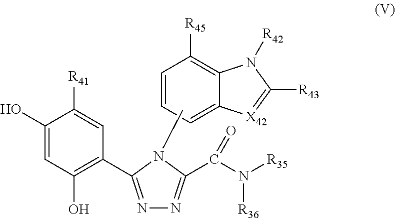
or a pharmaceutically acceptable salt thereof.

7. The method of claim 2, wherein the compound is according to the following formula (VI): 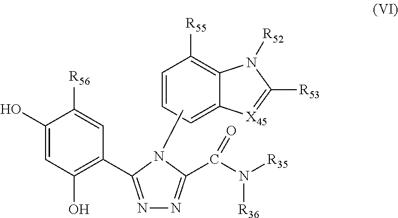
or a pharmaceutically acceptable salt thereof; wherein:
X₄₅ is CR₅₄ or N;
R₅₆ is selected from the group consisting of —H, methyl, ethyl, isopropyl, and cyclopropyl;
R₅₂ is selected from the group consisting of —H, methyl, ethyl, n-propyl, isopropyl, n-butyl, n-pentyl, n-hexyl, —(CH₂)₂OCH₃, —CH₂C(O)OH, and —C(O)N(CH₃)₂;
R₅₃ and R₅₄ are each, independently, —H, methyl, ethyl, or isopropyl; or
R₅₃ and R₅₄ taken together with the carbon atoms to which they are attached form a phenyl, cyclohexenyl, or cyclooctenyl ring; and
R₅₅ is selected from the group consisting of —H, —OH, —OCH₃, and OCH₂CH₃.

8. The method of claim 5, wherein the compound is according to the following formula (VII): 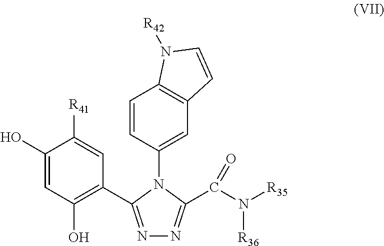
or a pharmaceutically acceptable salt thereof.

9. The method of claim 2, wherein the compound is selected from the group consisting of 5-(2,4-dihydroxy-5-isopropylphenyl)-N-(2-morpholinoethyl)-4-(4-(morpholinomethyl)phenyl)-4H-1,2,4-triazole-3-carboxamide; 5-(2,4-dihydroxy-5-isopropylphenyl)-4-(4-(morpholinomethyl)phenyl)-N-(5-sulfamoylpentyl)-4H-1,2,4-triazole-3-carboxamide; 5-(2,4-dihydroxy-5-isopropylphenyl)-N-(2-morpholinoethyl)-4-(4-morpholinophenyl)-4H-1,2,4-triazole-3-carboxamide; 5-(2,4-dihydroxy-5-isopropylphenyl)-4-(4-(morpholinomethyl)phenyl)-N-(2-(pyrrolidin-1-yl) ethyl)-4H-1,2,4-triazole-3-carboxamide; 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide; and 5-(2,4-dihydroxy-5-isopropylphenyl)-4-(4-((4-ethylpiperazin-1-yl)methyl)phenyl)-N-(2,2,2-trifluoroethyl)-4H-1,2,4-triazole-3-carboxamide, or a pharmaceutically acceptable salt thereof.

10. The method of claim 2, wherein the Hsp90 inhibitor is administered in combination with one or more additional therapeutic agents.

11. The method of claim 10, wherein the one or more additional therapeutic agents are selected from the group consisting of benazepril, captopril, enalapril, fosinopril, lisinopril, moexipril, perindopril, quinapril, ramipril, trandolapril, candesartan, eprosartan, irbesartan, telmisartan, valsartan, losartan, olmesartan, tolvaptan, lixivaptan, mozavaptan, SKI-606, sirolimus, rapamycin, and satavaptan.

12. The method of claim 11, wherein the additional therapeutic agent is tolvaptan.

13. The method of claim 11, wherein the additional therapeutic agent is lisinopril.

14. The method of claim 11, wherein the additional therapeutic agents are tolvaptan and lisinopril.

15. The method of claim 9, wherein the compound is 5-(2,4-dihydroxy-5-isopropylphenyl)-N-(2-morpholinoethyl)-4-(4-(morpholinomethyl)phenyl)-4H-1,2,4-triazole-3-carboxamide, or a pharmaceutically acceptable salt thereof.

16. The method of claim 15, wherein the Hsp90 inhibitor is administered in combination with one or more additional therapeutic agents.

17. The method of claim 16, wherein the one or more additonal therapeutic agents are selected from the group consisting of benazepril, captopril, enalapril, fosinopril, lisinopril, moexipril, perindopril, quinapril, ramipril, trandolapril, candesartan, eprosartan, irbesartan, telmisartan, valsartan, losartan, olmesartan, tolvaptan, lixivaptan, mozavaptan, SKI-606, sirolimus, rapamycin, and satavaptan.

18. The method of claim 17, wherein the additional therapeutic agent is tolvaptan.

19. The method of claim 17, wherein the additional therapeutic agent is lisinopril.

20. The method of claim 17, wherein the additional therapeutic agents are tolvaptan and lisinopril.
